(19) United States
(12) Patent Application Publication
(10) Pub. No.: US 2026/0272917 A1
(43) Pub. Date: Sep. 17, 2026

(54) COMPOSITIONS AND METHODS FOR DETECTING AND TREATING CANCER

(71) Applicant: CASE WESTERN RESERVE UNIVERSITY, Cleveland, OH (US)

(72) Inventors: Susann Brady-Kalnay, Wellington, OH (US); Kathleen Molyneaux, Cleveland, OH (US); Christian Laggner, Cleveland, OH (US)

(21) Appl. No.: 18/868,988

(22) PCT Filed: May 24, 2023
(86) PCT No.: PCT/US2023/023430

§ 371 Date: Nov. 25, 2024

Related U.S. Application Data
(60) Provisional application No. 63/345,100, filed on May 24, 2022.

(51) Int. Cl. A61K 31/497; A61K 31/42; A61K 31/423; A61K 31/445; A61K 31/454; A61K 31/4545; A61K 31/506; A61K 31/517; A61P 35/00

(52) U.S. Cl. CPC A61K 31/497; A61K 31/42; A61K 31/423; A61K 31/445; A61K 31/454; A61K 31/4545; A61K 31/506; A61K 31/517; A61P 35/00

(57) ABSTRACT

A pharmaceutical composition comprising a compound having the structure of formula (I) that targets receptor protein tyrosine phosphatase (RPTP) IIb cell adhesion molecules (e.g., PTPμ) and that is capable of inhibiting RPTP mediated adhesion of cells and/or cancer cell growth and/or sphere formation.

### RELATED APPLICATION

This application claims priority from U.S. Provisional Application No. 63/345,100, filed May 24, 2022, the subject matter of which is incorporated herein by reference in its entirety.

### BACKGROUND

Cancer detection and treatment are hindered by the inability to differentiate between cancer cells and normal cells. Better detection tools for cancer or tumor imaging are needed for earlier diagnosis of cancers. Molecular recognition of tumor cells would facilitate guided surgical resection. In order to improve surgical resection, targeted imaging tools must specifically label tumor cells, not only in the main tumor but also along the edge of the tumor and in the small tumor cell clusters that disperse throughout the body.

Targeted imaging tools designed to label molecules that accumulate in the tumor microenvironment may also be advantageous as therapeutic targeting agents, as they can identify both the main tumor cell population and areas with infiltrating cells that contribute to tumor recurrence. The ability to directly target the tumor cell and/or its microenvironment would increase both the specificity and sensitivity of current treatments, therefore reducing non-specific side effects of chemotherapeutics that affect cells throughout the body.

### SUMMARY

Embodiments described herein relate to compounds that target receptor protein tyrosine phosphatase (RPTP) cell adhesion molecules (e.g., PTPu) and that are capable of inhibiting RPTP mediated adhesion of cells and/or cancer cell growth and/or sphere formation as well as to their use in methods of detecting, monitoring, and/or imaging cancer cells and/or cancer cell metastasis, migration, dispersal, and/or invasion in a subject and methods of treating cancer in a subject in need thereof.

In some embodiments, the compound can have the structure of formula (I):
U-V-W-X-Y (I) or a pharmaceutically acceptable salt, tautomer, or solvate thereof, wherein,
U is cycloalkyl, aryl, heteroaryl, or heterocyclyl, each of which is optionally substituted with one or more R¹⁴;
V is absent, alkylene, —C(O)—, —C(O)—N(R¹⁵)₂—, or —N(R¹⁵)₂—C(O)—, -alkylene-C(O)—N(R¹⁵)₂—, or —N(R¹⁵)₂—C(O)-alkylene-;
W is alkylene, heterocyclene, -heterocyclene-alkylene-N(R¹⁵)₂—, or arylene, each of which is optionally substituted with one or more R¹⁶;
X is absent or S(O)ₙ;
Y is alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy;
each R¹⁷ is independently halogen, hydroxyl, alkyl, alkoxy, haloalkyl, cycloalkyl, aryl, heteroaryl, or heterocyclyl;
n is 0, 1, or 2; and
t is 0, 1, or 2.

In some embodiments, U is 5- to 10-membered heterocyclyl, 5- to 10-membered heteroaryl, or 6- to 10 membered aryl, each of which is optionally substituted with one or more R¹⁴.

In other embodiments, U is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In some embodiments, W is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In some embodiments, Y is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, the compound can have the structure of formula (IIA) or (IIB),

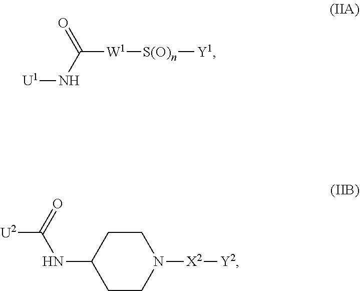

or a pharmaceutically acceptable salt, tautomer, or solvate thereof, wherein,
each of U1 or U2 is independently cycloalkyl, aryl, heteroaryl, or heterocyclyl, each of which is optionally substituted with one or more R¹⁴;
W¹ is alkylene, heterocyclene, heterocyclene-alkylene-N(R¹⁵)₂—, or arylene, each of which is optionally substituted with one or more R¹⁶;
X² is absent or S(O)ₙ;
each of Y¹ or Y² is independently alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy;
each R¹⁷ is independently halogen, hydroxyl, alkyl, alkoxy, haloalkyl, cycloalkyl, aryl, heteroaryl, or heterocyclyl;
n is 0, 1, or 2; and
t is 0, 1, or 2.

In some embodiments, U¹ or U² is 5- to 10-membered heterocyclyl, 5- to 10-membered heteroaryl, or 6- to 10 membered aryl, each of which is optionally substituted with one or more R¹⁴.

In other embodiments, U¹ or U² is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In some embodiments, W¹ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In some embodiments, Y¹ or Y² is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, the compound can have the structure of formula (IIIA):

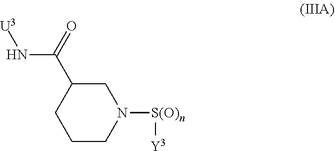

or a pharmaceutically acceptable salt, tautomer, or solvate thereof, wherein,
U³ is cycloalkyl, aryl, heteroaryl, or heterocyclyl, each of which is optionally substituted with one or more R¹⁴;
Y³ is alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, U³ is 5- to 10-membered heterocyclyl, 5- to 10-membered heteroaryl, or 6- to 10 membered aryl, each of which is optionally substituted with one or more R¹⁴

In other embodiments, U³ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In some embodiments, Y³ is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, the compound can have the structure of formula (IIIB):

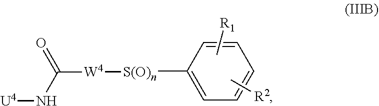

or a pharmaceutically acceptable salt, tautomer, or solvate thereof, wherein,
U⁴ is cycloalkyl, aryl, heteroaryl, or heterocyclyl, each of which is optionally substituted with one or more R¹⁴;
W⁴ is alkylene, heterocyclene, heterocyclene-alkylene-N(R¹⁵)₂—, or arylene, each of which is optionally substituted with one or more R¹⁶;
each of R¹ or R² is independently hydrogen, halogen, alkyl, haloalkyl, or alkoxy;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy; and
n is 0, 1, or 2.

In some embodiments, U⁴ is 5- to 10-membered heterocyclyl, 5- to 10-membered heteroaryl, or 6- to 10 membered aryl, each of which is optionally substituted with one or more R¹⁴.

In other embodiments, U⁴ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In some embodiments, W⁴ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In other embodiments, the compound can have the structure of formula (IIIC)

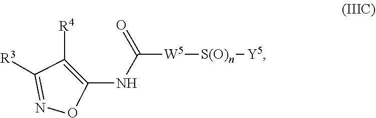

or a pharmaceutically acceptable salt, tautomer, or solvate thereof, wherein
W⁵ is alkylene, heterocyclene, heterocyclene-alkylene-N(R¹⁵)₂—, or arylene, each of which is optionally substituted with one or more R¹⁶;
Y⁵ is alkyl, cycloalkyl, aryl, heteroaryl, heterocyclyl, each of which is optionally substituted with one or more R¹⁷
each of R³ or R⁴ is independently hydrogen, halogen, alkyl, or haloalkyl, or alternatively R³ and R⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, W⁵ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In some embodiments, Y⁵ is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, the compound can have the structure of formula (IIID):

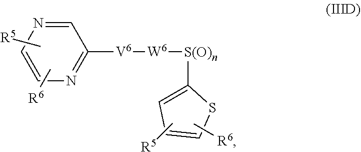

or a pharmaceutically acceptable salt, tautomer, or solvate thereof, wherein,
V⁶ is absent, alkylene, —C(O)—, —C(O)—N(R¹⁵)₂—, or —N(R¹⁵)₂—C(O)—, -alkylene-C(O)—N(R¹⁵)₂—, or —N(R¹⁵)₂—C(O)-alkylene-;
W⁶ is alkylene, heterocyclene, heterocyclene-alkylene-N(R¹⁵)₂—, or arylene, each of which is optionally substituted with one or more R¹⁶;
each of R⁵ or R⁶ is independently hydrogen, halogen, alkyl, haloalkyl, or —CN, or alternatively R⁵ and R⁶ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, or alkoxy; and
n is 0, 1, or 2.

In some embodiments, W⁶ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In other embodiments, the compound can have the structure of formula (IIIE):

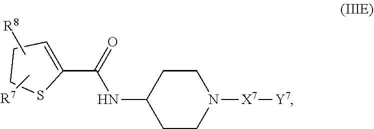

or a pharmaceutically acceptable salt, tautomer, or solvate thereof, wherein, X¹ is absent or S(O)ₙ;
Y⁷ is independently alkyl, cycloalkyl, aryl, heteroaryl, heterocyclyl, each of which is optionally substituted with one or more R¹⁷;
each of R⁷ or R⁸ is independently hydrogen, halogen, alkyl, or haloalkyl, or alternatively R⁷ and R⁸ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, Y⁷ is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, the compound can have the structure of formula (IIIF):

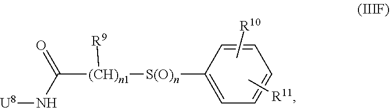

or a pharmaceutically acceptable salt, tautomer, or solvate thereof, wherein,
U⁸ is independently cycloalkyl, aryl, heteroaryl, or heterocyclyl, each of which is optionally substituted with one or more R¹⁴;
R⁹ is H, halogen, hydroxyl, alkyl, alkoxy;
each of R¹⁰ or R¹¹ is independently hydrogen, halogen, alkyl, haloalkyl, or —CN, or alternatively R¹⁰ and R¹¹ together with the atom(s) to which they are attached can form a 4- to 7-membered cycloalkyl, aryl, or heterocycle, optionally containing an additional heteroatom selected from O, S(O)ₜ, or N;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, U⁸ is 5- to 10-membered heterocyclyl, 5- to 10-membered heteroaryl, or 6- to 10 membered aryl, each of which is optionally substituted with one or more R¹⁴.

In other embodiments, U⁸ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In other embodiments, the compound can have the structure of formula (IIIA):

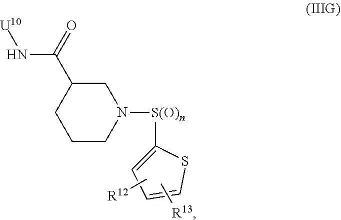

or a pharmaceutically acceptable salt, tautomer, or solvate thereof, wherein,
U¹⁰ is independently cycloalkyl, aryl, heteroaryl, or heterocyclyl, each of which is optionally substituted with one or more R¹⁴;
each of R¹² or R¹³ is independently hydrogen, halogen, alkyl, or haloalkyl, or alternatively R¹² and R¹³ together with the atom(s) to which they are attached can form a 4- to 7-membered cycloalkyl, aryl, or heterocycle, optionally containing an additional heteroatom selected from O, S(O)ₜ, or N;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N; and
n is 0, 1, or 2.

In some embodiments, U¹⁰ is 5- to 10-membered heterocyclyl, 5- to 10-membered heteroaryl, or 6- to 10 membered aryl, each of which is optionally substituted with one or more R¹⁴.

In other embodiments, U¹⁰ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In some embodiments, the compound can be formulated in a pharmaceutical composition with a pharmaceutically acceptable carrier or excipient.

In some embodiments, the compound specifically binds to and/or complexes with an extracellular fragment or portion of an RPTP cell adhesion molecule, such as PTPu, that is expressed by a cancer cell or another cell in the cancer cell microenvironment.

In some embodiments, the composition can be for use in detecting, monitoring, and/or imaging cancer cells and/or cancer cell metastasis, migration, dispersal, and/or invasion, and/or for treating cancer in a subject.

In other embodiments, the compositions is configured for in vivo administration to a subject or ex vivo administration to biological sample of the subject.

In some embodiments, the compound further includes a detectable moiety linked to and/or complexed with the compound. The detectable moiety can include, for example, at least one of a contrast agent, imaging agent, radiolabel, semiconductor particle, or nanoparticle.

In some embodiments, the detectable moiety is detectable by at least one of magnetic resonance imaging positron emission tomography (PET) imaging, computer tomography (CT) imaging, gamma imaging, near infrared imaging, or fluorescent imaging.

### BRIEF DESCRIPTION OF THE DRAWINGS

FIGS. 1(A-C) illustrate computational and functional screening strategy. (A) Shape overview of the PTPμ trans homodimer (PDB ID 2V5Y, white to gray with increasing residue number), with the location of the targeted binding sites circled. (B) Closeup of a monomer chain (region circled in (A)), showing the cleft between the MAM (left side, residues 22-184) and Ig (right side, residues 186-277) domains that was targeted as a potential ligand binding site. Binding site residues are shown for carbon, oxygen, and nitrogen atoms, respectively, with labels for the relevant amino acid residues. The residues were renumbered from the original PDB file to match the canonical sequence for UniProt ID P28827. (C) Functional screening approach. Atomwise provided 76 potential compounds and two blinded DMSO control samples. These were screened in cell-based assays for PTPu-mediated aggregation of Sf9 cells and glioma cell (LN229) sphere formation and growth in 3D culture. A third unblinded DMSO sample was used for normalization purposes. Compounds were identified that affected either or both assays.

FIG. 2 illustrates functional screening identifies compounds able to affect PTPμ-mediated aggregation of Sf9 cells. PTPμ-expressing Sf9 cells were treated with the indicated compounds (100 μM) for 20 min then rotated for 30 min to stimulate aggregation. (Top): images of samples treated with priority compounds. Each well was imaged in its entirety as a 4×4 grid, but just central tiles are shown. The examples show the replicate with the strongest effect (0205629321: n=8 and 0205603181: n=4). (Bottom): the number of aggregates with footprint areas over 4000 μm2 were counted and normalized to the average number present in the un-blinded DMSO control. The compound barcodes are shown on the x-axis. Inhibitors (<60%), activators (>120%), and the positions of the blinded DMSO samples are indicated. Colored asterisks indicate the functional categories of the compounds. Large asterisks indicate the top hit in each category. Values are averages±standard errors of the means (s.e.m). Samples that showed no effect in the initial screen (n=2). Priority compounds n=4-8 replicates.

FIG. 3 illustrates titration of selected compounds in the PTPμ aggregation assay in Sf9 cells. PTPμ-expressing cells were treated with the indicated doses of compounds then rotated to stimulate aggregation. (Left): examples of the effects of our two priority compounds at an intermediate dose (50 μM). Each well was imaged in its entirety as a 4×4 grid, but just representative central tiles are shown. (Right): quantitation of the dose-dependent effects of selected compounds on PTPμ aggregation in Sf9 cells. PTPμ-expressing cells were treated with the indicated doses of compounds then rotated to stimulate aggregation. The number of aggregates were counted and normalized to the average number present in the matched DMSO control. The initial hit (at 100 μM; n=2 replicates) and independent follow-up titration results (25-100 μM) are shown. Colored asterisks indicate the functional categories of the compounds. The large asterisks indicate the top hit in each category. Values are averages (n=2 replicates)±s.e.m.

FIG. 4 illustrates functional screening identifies compounds able to inhibit glioma sphere formation and growth. LN229 cells were plated into non-adherent 96-well u-bottom plates and treated with compounds (100 μM) for 7 days. (Left): representative images of day 1 and day 7 spheres treated with the priority compounds (100 μM). On day 1, a larger footprint size indicates that sphere formation was slowed. On day 7, a smaller size change indicates impaired growth. (Right): quantitation of the effects of all soluble non-toxic compounds (100 μM) on sphere formation and growth. Sphere footprint sizes were measured on day 1 and normalized to the average footprint size of the un-blinded DMSO controls. Sphere footprint sizes were measured again on day 7 and the % size change calculated and normalized to the average size change of the controls. Inhibitors and the positions of the blinded DMSO samples are indicated. Colored asterisks indicate the functional categories of the compounds. The large asterisks indicate the top hit in each category. Values are averages±s.e.m. Samples that showed no effect in the initial screen (n=2). Priority compounds n=4-8 replicates.

FIG. 5 illustrates titration of the effects of selected compounds on sphere formation and growth. LN229 cells were plated into non-adherent 96-well u-bottom plates and treated with the indicated doses of compounds for 7 days. (Left): representative images of day 1 and day 7 spheres treated with the two priority compounds. (Right): quantitation of the dose-dependent effects of selected compounds on glioma sphere formation and growth. Sphere footprint sizes were measured on day 1 and normalized to the average footprint size of the un-blinded DMSO controls. Sphere footprint sizes were measured again on day 7 and the % size change calculated and normalized to the average size change of the controls. The initial hit (at 100 μM) and independent follow-up titration results (25-100 μM) are shown. Colored asterisks indicate the functional category of the compound. The large asterisks indicate the top hit in each category. Values are averages (n=2 replicates)±s.e.m.

FIG. 6 illustrate the effect of selected compounds in a bead-based aggregation assay. MonoMag streptavidin beads coated with PTPμ21-365avi were treated with 100 μM compounds for 20 min and then induced to aggregate by rotation. The number of aggregates over 50 μm2 in 16 frames per well were counted and normalized to the average number present in the vehicle-treated control wells. Colored asterisks indicate the functional category of the compound. Values are averages±s.e.m. Significance was tested with Student's t-test. p-values are shown for those compounds found to cause a statistically significant effect (p<0.05) on aggregation (relative to the DMSO control).

FIG. 7 illustrates examples of Helix Blue staining for cell toxicity. LN229 cells (plated onto non-adherent surfaces) or Sf9 cells were treated for 24 h with 100 μM of the indicated compounds. Two examples of untreated glioma spheres are shown to illustrate the varying degree of cell-death present in normal spheres. The two priority compounds identified in our screen were not toxic to either cell type. One compound was found to be toxic to both cell types and was eliminated from the screen.

FIG. 8 illustrates examples of insoluble compounds. Eighteen compounds were eliminated due to signs of insolubility in either/both the aggregation assay or sphere assay. The appearance of precipitate varied depending on the compound.

FIG. 9 illustrates structure and best predicted binding pose for a carbon base structure of 0205629321. Atoms in the carbon base structure of 0205629321 were docked with mCule 1-click docking (https://mcule.com/apps/1-click-docking/) using the crystal structure of the extracellular domain (Protein Data Bank IDs 2V5Y) and entry to the binding pocket defined as His187. The predicted Gibb's free energy for association of 0205629321 was-7.6.

FIG. 10 illustrates structure and best predicted binding pose for a carbon base structure of 0205603181. Atoms in the carbon base structure of 0205603181 were docked with mCule 1-click docking using the crystal structure of the extracellular domain (Protein Data Bank IDs 2V5Y) and entry to the binding pocket defined as His187. The predicted Gibb's free energy for 0205603181 was-6.6.

FIG. 11 illustrates titration of two compounds that inhibited PTPμ aggregation in Sf9 cells but did not affect glioma cell spheres. PTPμ-expressing Sf9 cells were treated with the indicated concentration of compounds for 20 min then rotated for 30 min to stimulate aggregation. Each well was imaged in its entirety as a 4×4 grid, but just representative central tiles are shown. The strongest inhibitor identified in the initial screen (FIG. 2) was active down to 25 μM.

FIG. 12 illustrates titration of one compound that stimulated PTPμ aggregation in Sf9 cells but did not affect glioma cell spheres. PTPμ-expressing Sf9 cells were treated with the indicated concentration of compounds for 20 min then rotated for 30 min to stimulate aggregation. Each well was imaged in its entirety as a 4×4 grid, but just representative central tiles are shown. A potential activator identified in the initial screen (FIG. 2) was weak on follow-up.

FIG. 13 illustrates titration of five compounds that inhibited glioma sphere formation/growth but did not affect PTPμ aggregation in Sf9 cells. LN229 cells were plated into non-adherent 96-well u-bottom plates and treated with the indicated doses of compounds. Sphere footprint sizes were measured on day 1 and sphere growth measured on day 7. One compound inhibited sphere formation down to 50 μM and, at 25 μM, altered the shape of day 7 spheres. One compound inhibited sphere formation at 100 μM and caused surface sloughing of cells in day 7 spheres at 100 and 50 μM. The other three compounds had only modest inhibitory effects on sphere growth at 100 μM on follow-up.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —OR^{a} where R^{a} is an alkyl, alkenyl or alknyl radical as defined above containing one to twelve carbon atoms. Unless stated otherwise specifically in the specification, an alkoxy group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R^{b}—R^{c} where R^{b} is an alkylene group as defined above and R^{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R^{b}—R^{c} where R^{b} is an alkenylene group as defined above and R^{c} is one or more aryl radicals as defined above. Unless stated otherwise specifically in the specification, an aralkenyl group can be optionally substituted.

“Aralkynyl” or “arylalkynyl” refers to a radical of the formula —R^{b}—R^{c} where R^{b} is an alkynylene group as defined above and R^{e} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R^{b}—R^{d} where R^{b} is an alkylene, alkenylene, or alkynylene group as defined above and R^{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R^{b}—R^{e} where R^{b} is an alkylene group as defined above and R^{e} is a heterocyclyl radical as defined above. Unless stated otherwise specifically in the specification, a heterocyclylalkyl group can be optionally substituted.

“Heterocyclylalkenyl” refers to a radical of the formula —R^{b}—R^{e} where R^{b} is an alkenylene group as defined above and R^{e} is a heterocyclyl radical as defined above. Unless stated otherwise specifically in the specification, a heterocyclylalkenyl group can be optionally substituted.

“Heterocyclylalkynyl” refers to a radical of the formula —R^{b}—R^{e} where R^{b} is an alkynylene group as defined above and R^{e} is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R^{b}—R^{f} where R^{b} is an alkylene chain as defined above and R^{f} is a heteroaryl radical as defined above. Unless stated otherwise specifically in the specification, a heteroarylalkyl group can be optionally substituted.

“Heteroarylalkenyl” refers to a radical of the formula —R^{b}—R^{f} where R^{b} is an alkenylene, chain as defined above and R^{f} is a heteroaryl radical as defined above. Unless stated otherwise specifically in the specification, a heteroarylalkenyl group can be optionally substituted.

“Heteroarylalkynyl” refers to a radical of the formula —R^{b}—R^{f} where R^{b} is an alkynylene chain as defined above and R^{f} is a heteroaryl radical as defined above. Unless stated otherwise specifically in the specification, a heteroarylalkynyl group can be optionally substituted.

“Thioalkyl” refers to a radical of the formula —SR^{a} where R^{a} is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom, such as oxygen in oxo, carbonyl, carboxyl, and ester groups; and nitrogen in groups such as imines, oximes, hydrazones, and nitriles. For example, “substituted” includes any of the above groups in which one or more hydrogen atoms with -NRgRh, -NRgC(═O) Rh, -NRgC(═O)NRgRh, -NRgC(═O) ORh, -NRgSO2Rh, —OC(═O)NR gRh, -ORg, -SRg, -SORg, -SO₂Rg, -OSO₂Rg, -SO₂ORg, =NSO₂Rg, and -SO₂NRgRh. “Substituted” also means any of the above groups in which one or more hydrogen atoms are replaced with —C(═O)Rg, —C(═O)ORg, —C(═O)NRgRh, —CH₂SO₂Rg, —CH₂SO₂NRgRh. In the foregoing, Rg and Rh are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

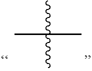

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

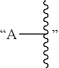

indicates that the chemical entity “A” is bonded to another chemical entity via the point of attachment bond. Furthermore, the specific point of attachment to the non-depicted chemical entity can be specified by inference. For example, the compound

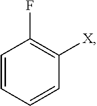

wherein X is “

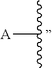

infers that the point of attachment bond is the bond by which X is depicted as being attached to the phenyl ring at the ortho position relative to fluorine.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Embodiments described herein relate to compounds that target receptor protein tyrosine phosphatase (RPTP) cell adhesion molecules (e.g., PTPμ) and that are capable of inhibiting RPTP mediated adhesion of cells and/or cancer cell growth and/or sphere formation as well as to their use in methods of detecting, monitoring, and/or imaging cancer cells and/or cancer cell metastasis, migration, dispersal, and/or invasion in a subject and methods of treating cancer in a subject in need thereof.

PTPμ is a member of the receptor protein tyrosine phosphatase IIb family that participates in cell-cell adhesion and signaling. PTPμ is proteolytically downregulated in glioblastoma (glioma), and the resulting extracellular and intracellular fragments are believed to stimulate cancer cell growth and/or migration. Compounds targeting these fragments can have therapeutic potential.

We used a deep learning neural network for drug design and discovery, to screen a molecular library of several million compounds and identified candidates predicted to interact with a groove between the MAM and Ig extracellular domains required for PTPμ-mediated cell adhesion. These candidates were then screened in two cell-based assays: PTPμ-dependent aggregation of Sf9 cells and a tumor growth assay where glioma cells grow in three-dimensional spheres. Compounds that inhibited PTPμ-mediated aggregation of Sf9 cells and/or inhibited glioma sphere formation/growth can be used, for example, as a targeted molecular imaging agent in brain tumor diagnosis, as a targeted optical imaging agent in fluorescent guided surgical resection of brain tumors, and as a therapeutic agent to antagonize the effect of the extracellular domain of PTPμ in gliobastoma to reduce invasion and metastasis.

For example, when the compound includes a detectable moiety that is directly or indirectly linked to the compound, the compound can demarcate tumor cells in tissue sections and tumor “edge” samples, suggesting that the compound can be used as a diagnostic tool for molecular imaging of metastatic, dispersive, migrating, or invading cancers or the tumor margin. Systemic introduction of compound as described herein can result in specific labeling of the tumors.

The compounds described herein can be administered systemically to a subject and readily target cancer cells associated with proteolytically cleaved extracellular fragment of the RPTP type IIb cell adhesion molecules, such as metastatic, migrating, dispersed, and/or invasive cancer cells. In some embodiments, the compounds after systemic administration can cross the blood brain barrier to define cancer cell location, distribution, metastases, dispersions, migrations, and/or invasion as well as tumor cell margins in the subject. In other embodiments, the compounds after systemic administration can inhibit and/or reduce cancer cell growth, survival, proliferation, and migration.

The compounds described herein can therefore be used in a method of detecting cancer cells and/or cancer cell metastasis, migration, dispersal, and/or invasion as well as in a method of treating cancer in a subject in need thereof. The methods can include administering to a subject a compound that binds to and/or complexes with the RPTP cell adhesion molecule in the cancer cell or tumor cell microenvironment. The compound bound to and/or complexed with the RPTP cell adhesion molecule expressed by the cancer cells can be detected to determine the location and/or distribution of the cancer cells in the subject as well as inhibit and/or reduce cancer cell growth, survival, proliferation, and migration.

In some embodiments, the compound can have the structure of formula (I):
U-V-W-X-Y (I) or a pharmaceutically acceptable salt, tautomer, or solvate thereof, wherein,
U is cycloalkyl, aryl, heteroaryl, or heterocyclyl, each of which is optionally substituted with one or more R¹⁴;
V is absent, alkylene, —C(O)—, —C(O)—N(R¹⁵)₂—, or —N(R¹⁵)₂—C(O)—, -alkylene-C(O)—N(R¹⁵)₂—, or —N(R¹⁵)₂—C(O)-alkylene-;
W is alkylene, heterocyclene, heterocyclene-alkylene-N(R¹⁵)₂—, or arylene, each of which is optionally substituted with one or more R¹⁶;
X is absent or S(O)ₙ;
Y is alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy;
each R¹⁷ is independently halogen, hydroxyl, alkyl, alkoxy, haloalkyl, cycloalkyl, aryl, heteroaryl, or heterocyclyl;
n is 0, 1, or 2; and
t is 0, 1, or 2.

In some embodiments, U is 5- to 10-membered heterocyclyl, 5- to 10-membered heteroaryl, or 6- to 10 membered aryl, each of which is optionally substituted with one or more R¹⁴.

In other embodiments, U is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In still other embodiments, U is

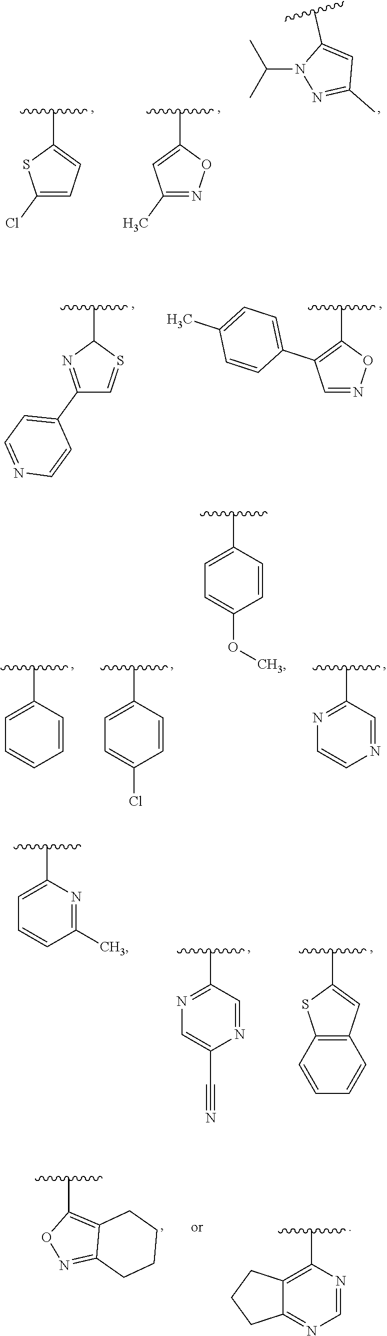

In some embodiments, W is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In some embodiments, Y is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, Y is

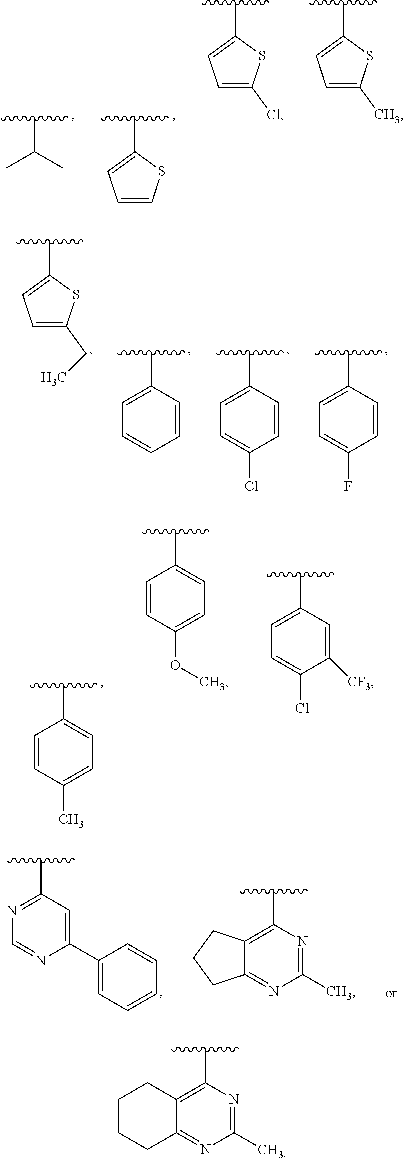

In other embodiments, the compound can have the structure of formula (IIA) or (IIB),

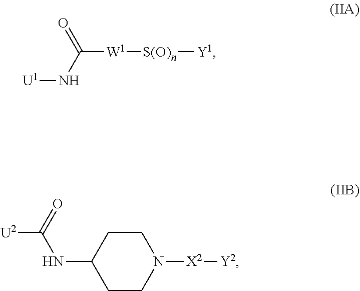

or a pharmaceutically acceptable salt, tautomer, or solvate thereof, wherein,
each of U¹ or U² is independently cycloalkyl, aryl, heteroaryl, or heterocyclyl, each of which is optionally substituted with one or more R¹⁴;
W¹ is alkylene, heterocyclene, heterocyclene-alkylene-N(R¹⁵)₂—, or arylene, each of which is optionally substituted with one or more R¹⁶;
X² is absent or S(O)ₙ;
each of Y¹ or Y² is independently alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy;
each R¹⁷ is independently halogen, hydroxyl, alkyl, alkoxy, haloalkyl, cycloalkyl, aryl, heteroaryl, or heterocyclyl;
n is 0, 1, or 2; and
t is 0, 1, or 2.

In some embodiments, U¹ or U² is 5- to 10-membered heterocyclyl, 5- to 10-membered heteroaryl, or 6- to 10 membered aryl, each of which is optionally substituted with one or more R¹⁴.

In other embodiments, U¹ or U² is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In still other embodiments, U¹ or U² is

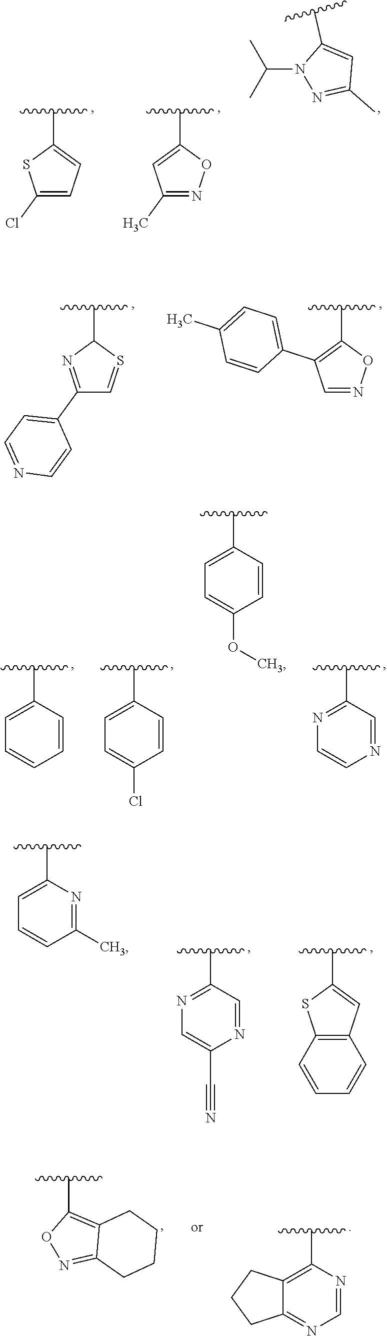

In some embodiments, W¹ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In some embodiments, Y¹ or Y² is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, Y¹ or Y² is

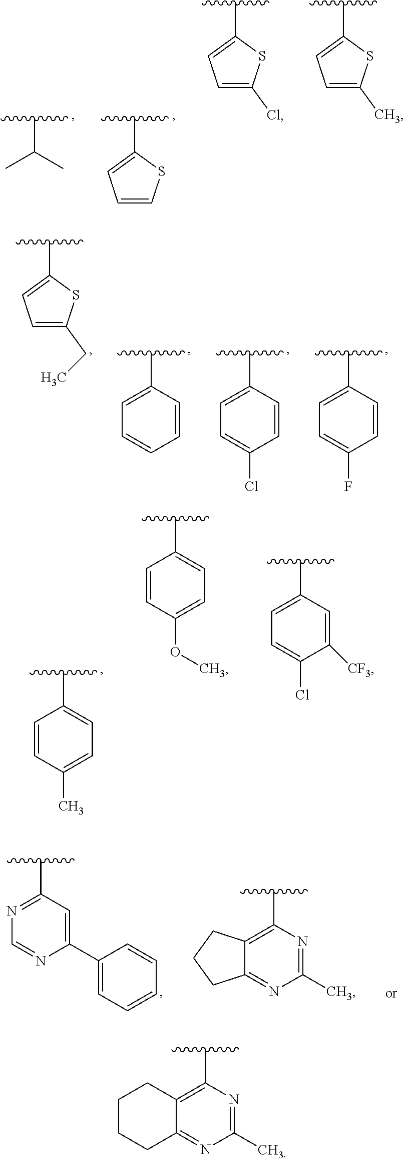

In other embodiments, the compound can have the structure of formula (IIIA):

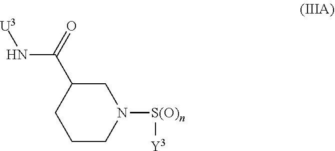

or a pharmaceutically acceptable salt, tautomer, or solvate thereof, wherein,
U³ is cycloalkyl, aryl, heteroaryl, or heterocyclyl, each of which is optionally substituted with one or more R¹⁴,
Y³ is alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, U³ is 5- to 10-membered heterocyclyl, 5- to 10-membered heteroaryl, or 6- to 10 membered aryl, each of which is optionally substituted with one or more R¹⁴.

In other embodiments, U³ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In still other embodiments, U³ is

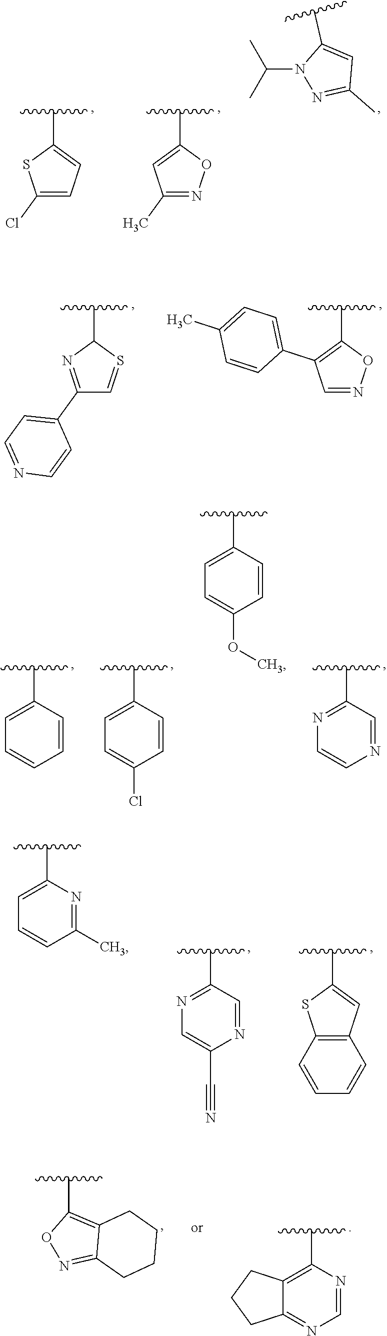

In some embodiments, Y³ is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, Y³ is

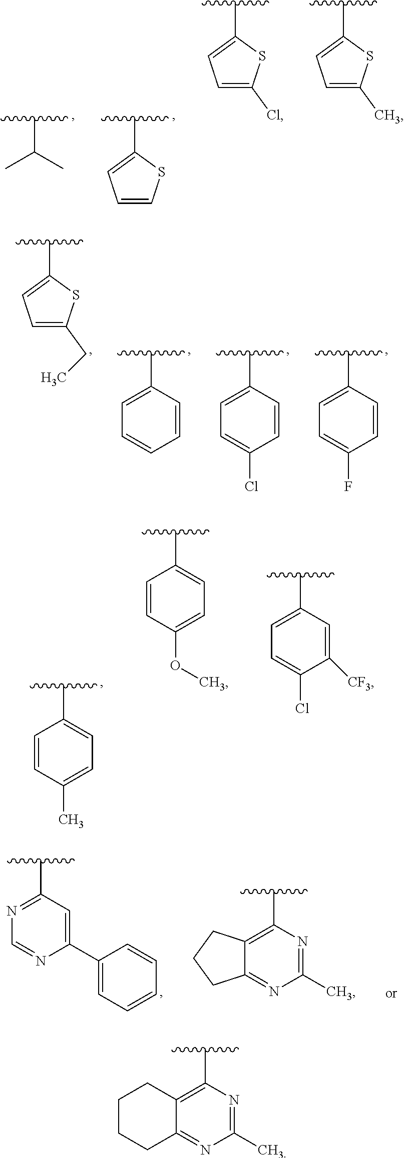

In other embodiments, the compound can have the structure of formula (IIIB):

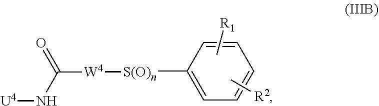

or a pharmaceutically acceptable salt, tautomer, or solvate thereof, wherein,
U⁴ is cycloalkyl, aryl, heteroaryl, or heterocyclyl, each of which is optionally substituted with one or more R¹⁴;
W⁴ is alkylene, heterocyclene, heterocyclene-alkylene-N(R¹⁵)₂—, or arylene, each of which is optionally substituted with one or more R¹⁶;
each of R¹ or R² is independently hydrogen, halogen, alkyl, haloalkyl, or alkoxy;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy; and
n is 0, 1, or 2.

In some embodiments, U⁴ is 5- to 10-membered heterocyclyl, 5- to 10-membered heteroaryl, or 6- to 10 membered aryl, each of which is optionally substituted with one or more R¹⁴.

In other embodiments, U⁴ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In still other embodiments, U⁴ is

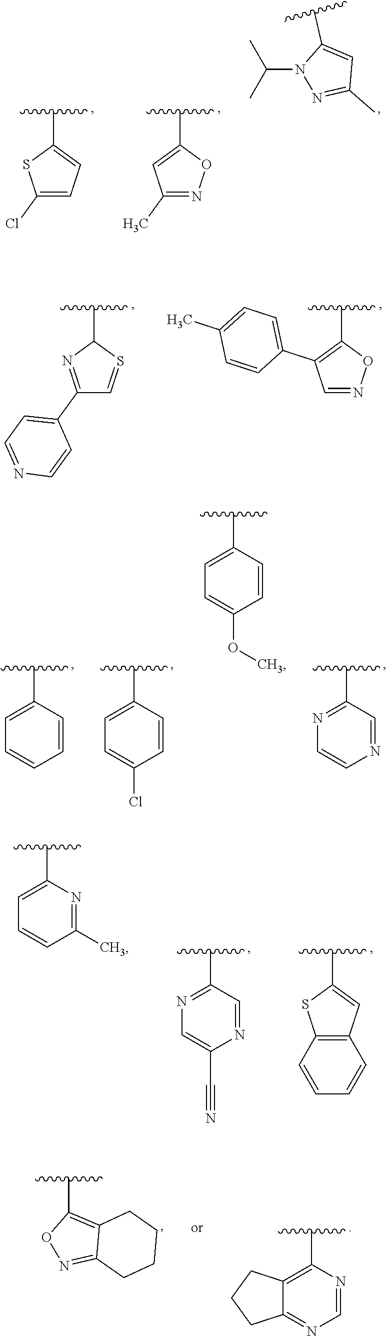

In some embodiments, W⁴ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In other embodiments, the compound can have the structure of formula (IIIC):

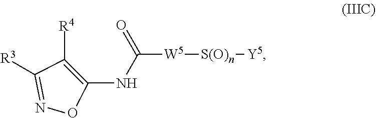

or a pharmaceutically acceptable salt, tautomer, or solvate thereof, wherein,
W⁵ is alkylene, heterocyclene, heterocyclene-alkylene-N(R¹⁵)₂—, or arylene, each of which is optionally substituted with one or more R¹⁶;
Y⁵ is alkyl, cycloalkyl, aryl, heteroaryl, heterocyclyl, each of which is optionally substituted with one or more R¹⁷;
each of R³ or R⁴ is independently hydrogen, halogen, alkyl, or haloalkyl, or alternatively R³ and R⁴ together with the atom(s) to which they are attached can form a 4- to 7-membered cycloalkyl, aryl, or heterocycle, optionally containing an additional heteroatom selected from O, S(O)ₜ, or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy;
each R¹⁷ is halogen, hydroxyl, alkyl, alkoxy, haloalkyl, cycloalkyl, aryl, heteroaryl, or heterocyclyl; and
n is 0, 1, or 2.

In some embodiments, W⁵ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In some embodiments, Y⁵ is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, Y⁵ is

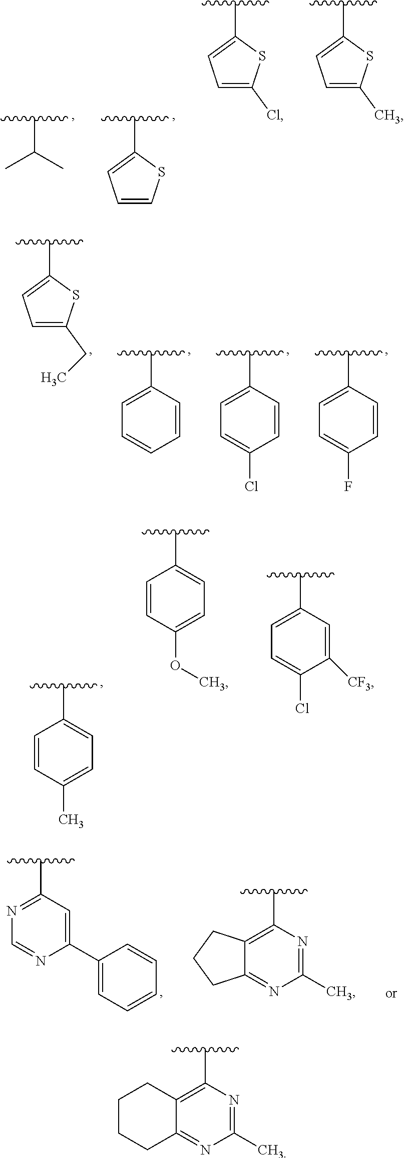

In other embodiments, the compound can have the structure of formula (IIID):

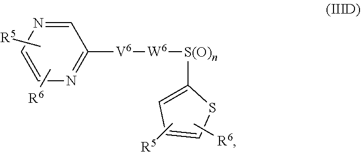

or a pharmaceutically acceptable salt, tautomer, or solvate thereof, wherein,
V⁶ is absent, alkylene, —C(O)—, —C(O)—N(R¹⁵)₂—, or —N(R¹⁵)₂—C(O)—, -alkylene-C(O)—N(R¹⁵)₂—, or —N(R¹⁵)₂—C(O)-alkylene-;
W⁶ is alkylene, heterocyclene, heterocyclene-alkylene-N(R¹⁵)₂—, or arylene, each of which is optionally substituted with one or more R¹⁶;
each of R⁵ or R⁶ is independently hydrogen, halogen, alkyl, haloalkyl, or —CN, or alternatively R⁵ and R⁶ together with the atom(s) to which they are attached can form a 4- to 7-membered cycloalkyl, aryl, heteroaryl, or heterocycle, optionally containing an additional heteroatom selected from O, S(O), or N;
each R¹⁵ is independently, H, alkyl, -alkylene-OH, —C(O)-alkyl, —C(O)O-alkyl, or -alkylene-COOH;
each R¹⁶ is independently halogen, hydroxyl, alkyl, haloalkyl, or alkoxy; and
n is 0, 1, or 2.

In some embodiments, W⁶ is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

In other embodiments, the compound can have the structure of formula (IIIE):

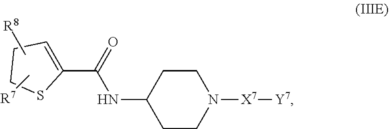

or a pharmaceutically acceptable salt, tautomer, or solvate thereof, wherein,
X⁷ is absent or S(O)ₙ;
Y⁷ is independently alkyl, cycloalkyl, aryl, heteroaryl, heterocyclyl, each of which is optionally substituted with one or more R¹⁷;
each of R⁷ or R⁸ is independently hydrogen, halogen, alkyl, or haloalkyl, or alternatively R⁷ and R⁸ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, Y⁷ is C₁-C₆ alkyl, 5- to 10-membered heterocyclyl, 5- to 10-membered heteroaryl, or 6- to 10 membered aryl, each of which is optionally substituted with one or more R¹⁷.

In other embodiments, Y⁷ is

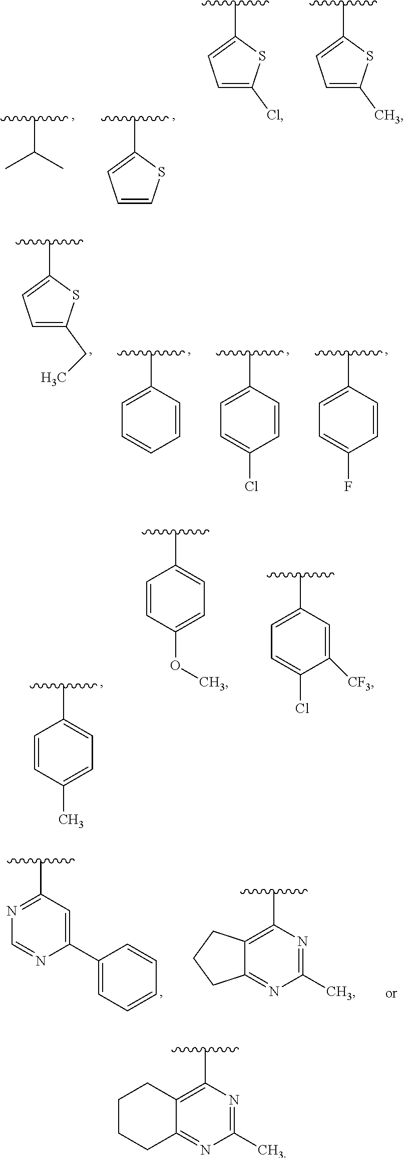

In other embodiments, the compound can have the structure of formula (IIIF):

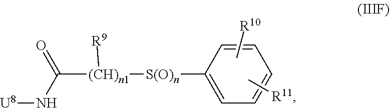

or a pharmaceutically acceptable salt, tautomer, or solvate thereof, wherein,
U⁸ is independently cycloalkyl, aryl, heteroaryl, or heterocyclyl, each of which is optionally substituted with one or more R¹⁴;
R⁹ is H, halogen, hydroxyl, alkyl, alkoxy;
each of R¹⁰ or R¹¹ is independently hydrogen, halogen, alkyl, haloalkyl, or —CN, or alternatively R¹⁰ and R¹¹ together with the atom(s) to which they are attached can form a 4- to 7-membered cycloalkyl, aryl, or heterocycle, optionally containing an additional heteroatom selected from O, S(O)ₜ, or N;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O), or N;
each R¹⁷ is independently halogen, hydroxyl, alkyl, alkoxy, haloalkyl, cycloalkyl, aryl, heteroaryl, or heterocyclyl; and
n is 0, 1, or 2.

In some embodiments, U⁸ is 5- to 10-membered heterocyclyl, 5- to 10-membered heteroaryl, or 6- to 10 membered aryl, each of which is optionally substituted with one or more R¹⁴.

In other embodiments, U⁸ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In still other embodiments, U⁸ is

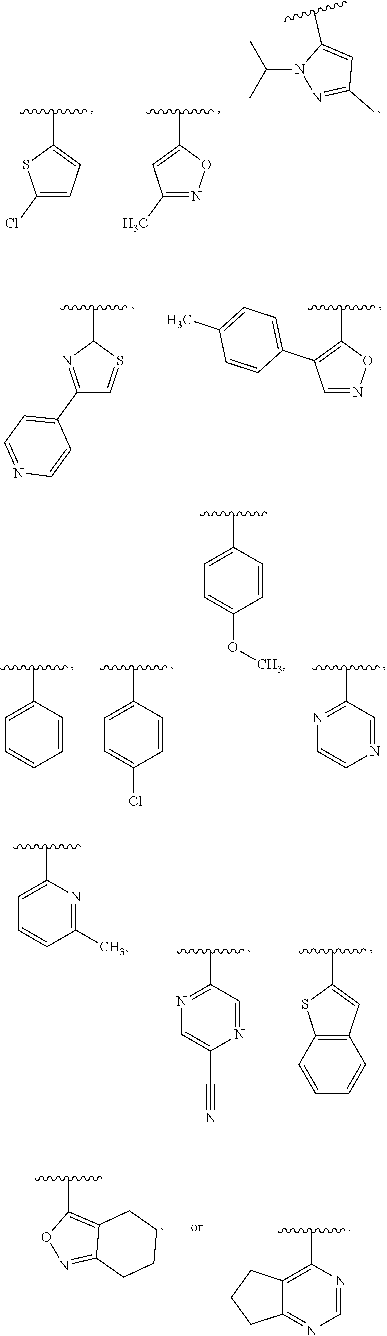

In other embodiments, the compound can have the structure of formula (IIIA):

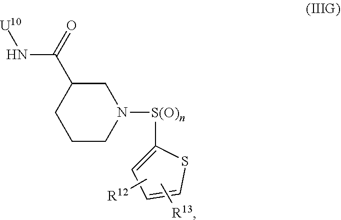

or a pharmaceutically acceptable salt, tautomer, or solvate thereof, wherein,
U¹⁰ is independently cycloalkyl, aryl, heteroaryl, or heterocyclyl, each of which is optionally substituted with one or more R¹⁴;
each of R¹² or R¹³ is independently hydrogen, halogen, alkyl, or haloalkyl, or alternatively R¹² and R¹³ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, or heterocycle, optionally containing an additional heteroatom selected from O, S(O)ₜ, or N; and
n is 0, 1, or 2.

In some embodiments, U¹⁰ is 5- to 10-membered heterocyclyl, 5- to 10-membered heteroaryl, or 6- to 10 membered aryl, each of which is optionally substituted with one or more R¹⁴.

In other embodiments, U¹⁰ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

In still other embodiments, U¹⁰ is

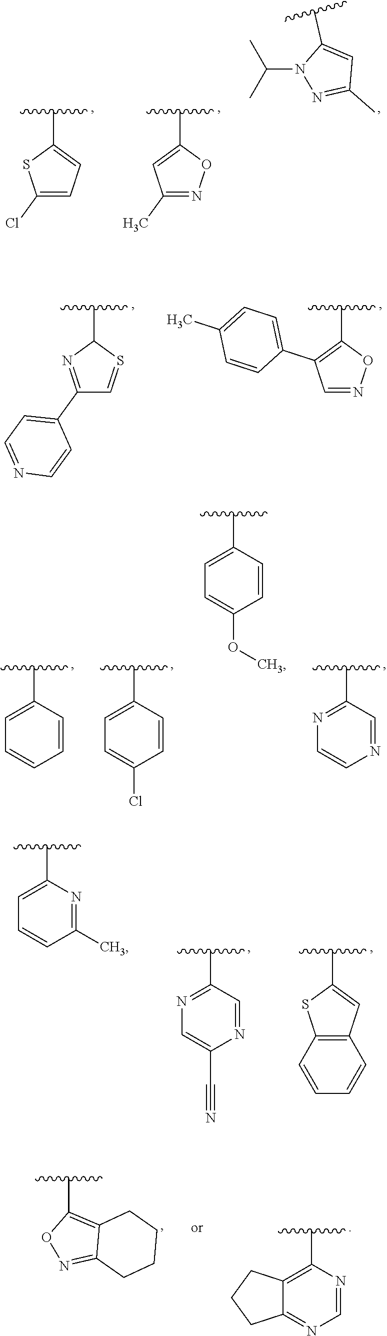

In other embodiments, the compound can have the structure of a formula selected from:

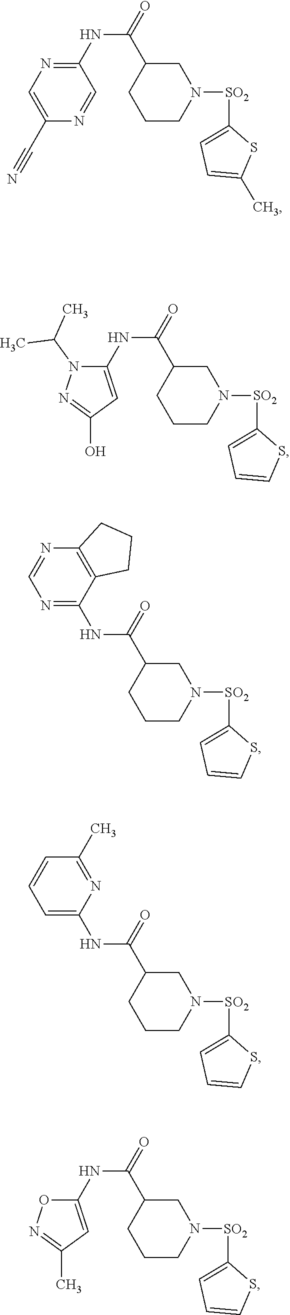 [SMILES: Cc1ccc(S(=O)(=O)N2CCCC(C(=O)Nc3cnc(C#N)cn3)C2)s1 ; CC(C)n1nc(O)cc1NC(=O)C1CCCN(S(=O)(=O)c2cccs2)C1 ; O=C(Nc1ncnc2c1CCC2)C1CCCN(S(=O)(=O)c2cccs2)C1 ; Cc1cccc(NC(=O)C2CCCN(S(=O)(=O)c3cccs3)C2)n1 ; Cc1cc(NC(=O)C2CCCN(S(=O)(=O)c3cccs3)C2)on1]

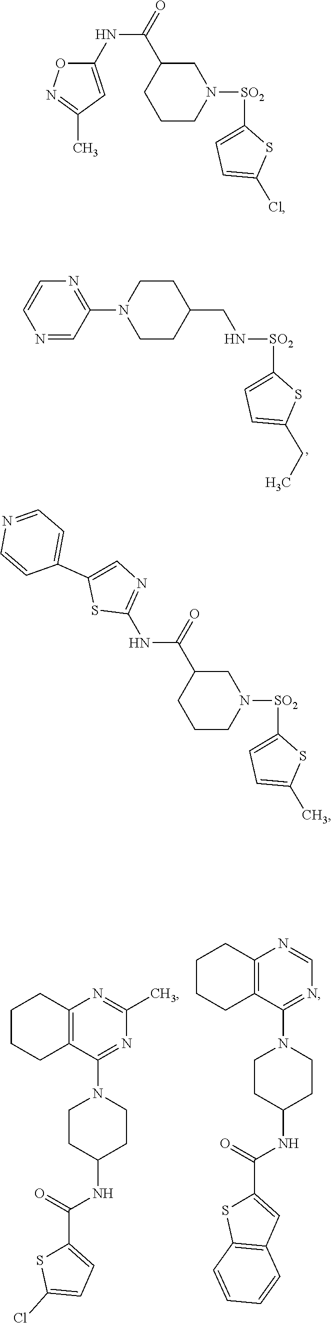

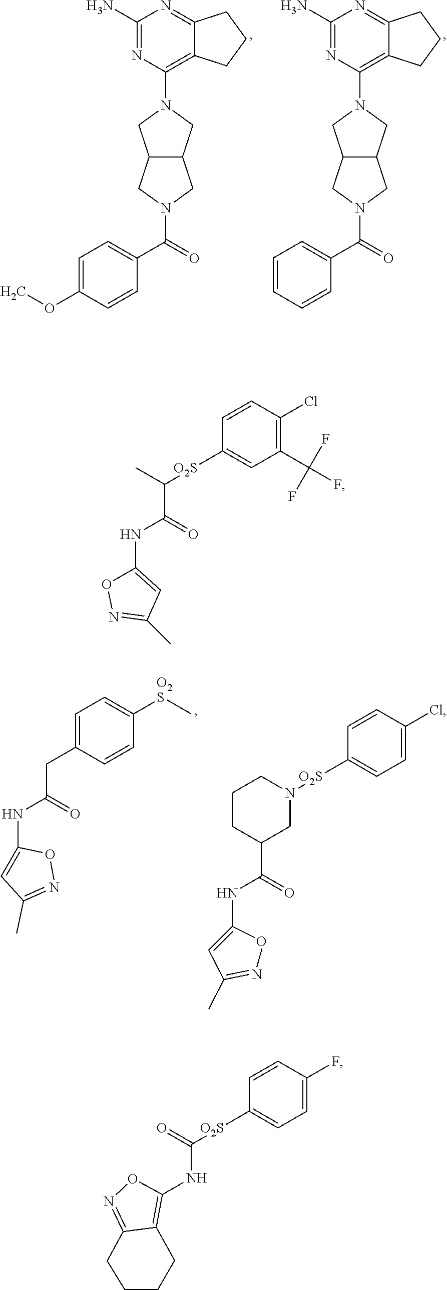 [SMILES: COc1ccc(C(=O)N2CC3CN(c4nc(N)nc5c4CCC5)CC3C2)cc1 ; Nc1nc2c(c(N3CC4CN(C(=O)c5ccccc5)CC4C3)n1)CCC2 ; Cc1cc(NC(=O)C(C)S(=O)(=O)c2ccc(Cl)c(C(F)(F)F)c2)on1 ; Cc1cc(NC(=O)Cc2ccc(S(C)(=O)=O)cc2)on1 ; Cc1cc(NC(=O)C2CCCN(S(=O)(=O)c3ccc(Cl)cc3)C2)on1 ; O=C(Nc1onc2c1CCCC2)S(=O)(=O)c1ccc(F)cc1]

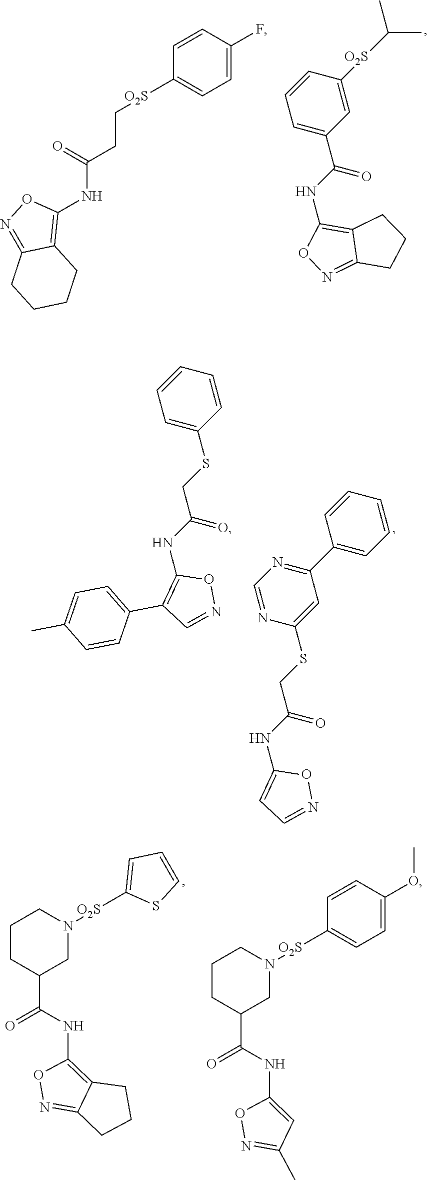

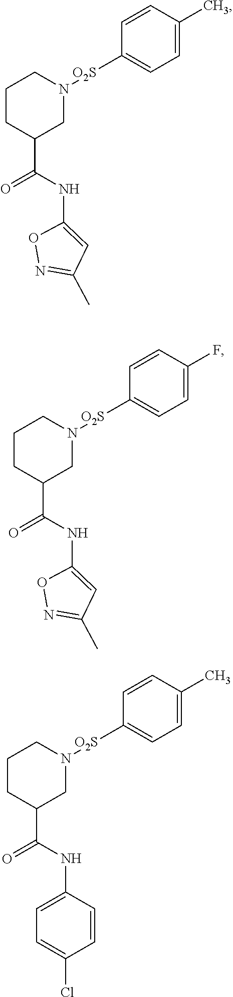 [SMILES: Cc1ccc(S(=O)(=O)N2CCCC(C(=O)Nc3cc(C)no3)C2)cc1 ; Cc1cc(NC(=O)C2CCCN(S(=O)(=O)c3ccc(F)cc3)C2)on1 ; Cc1ccc(S(=O)(=O)N2CCCC(C(=O)Nc3ccc(Cl)cc3)C2)cc1]

or a pharmaceutically acceptable salt, tautomer, or solvate thereof.

In some embodiments, the efficacy of the compound can be assessed by generating dose response curves from data obtained using various concentrations of a test compound. Moreover, a control assay can also be performed to provide a baseline for comparison. Such candidates can be further tested for efficacy in inhibiting chemotaxis of cancer cells in vitro, spreading, invasion, or migration of cancer cells in vitro, for efficacy in tumor dispersal, or spreading in vitro or in vivo. For example, the efficacy of the compound can be tested in vivo in animal cancer models.

Cell-based assays may be performed as either a primary screen, or as a secondary screen to confirm the activity of agents identified in a cell free screen, such as an in silico screen. Such cell based assays can employ a cell-type expressing the RPTP. Exemplary cell types include cancer cell lines, primary tumor xenografts, and glioma cells. Cells in culture are contacted with one or more compounds, and the ability of the one or more compounds to inhibit cell migration/invasion is measured. Compounds that inhibit cell migration/invasion are candidate compounds for use in the subject methods of inhibiting tumor progression. For example, the identified compounds can be tested in cancer models known in the art.

In some embodiments, efficacy of the compound can be measured using a PTPμ-dependent Sf9 aggregation assay. The Sf9 assay directly tests adhesive action of PTPμ since Sf9 cells lack endogenous PTPμ and do not normally self-aggregate. However, baculoviral-mediated overexpression of PTPμ drives homophilic adhesion of Sf9 cells on non-adhesive coated wells. PTPμ expressing Sf9 cells readily aggregate in control samples, but wells treated with therapeutically effective compounds can contain mostly single cells or small clusters. In some embodiments, aggregation of PTPμ expressing Sf9 cells administered about 100 μM, preferably about 50 μM, or more preferably about 25 μM, of a compound described herein (e.g., formula I, IIA, IIB, and IIIA-IIIG) is less than 60%, less than 55%, less than 50%, less than 45%, less than 40%, less than 35%, less than 30%, or less than 25% compared to PTPμ expressing SFF9 cells administered DMSO.

In other embodiments, efficacy of the compound can be measured using a glioma sphere assay. This assay was selected to run in parallel because the ultimate goal is to identify compounds that have therapeutic potential against cancers such as glioblastoma. Glioma cells (LN229s) cultured on non-adhesive coating cluster together and grow as 3D structures that can model some of the complexity of the tumor microenvironment. In some embodiments, about 100 μM, preferably about 50 μM, or more preferably about 25 μM of a compound described herein (e.g., formula I, IIA, IIB, and IIIA-IIIG) can inhibits aggregation of glioma sphere formation compared glioma cells administered DMSO.

In some embodiments, the compounds can include or be directly or indirectly coupled to a detectable moiety. The detectable moiety can include any contrast agent or detectable label that facilitate the detection step of a diagnostic or therapeutic method by allowing visualization of the complex formed by binding of the compound to the RPTP cell adhesion molecule. The detectable moiety can be selected such that it generates a signal, which can be measured and whose intensity is related (preferably proportional) to the amount of the compound bound to the tissue being analyzed.

Any of a wide variety of detectable moieties can be linked with the compound described herein. Examples of detectable moieties include, but are not limited to: various ligands, radionuclides, fluorescent agents and dyes, infrared and near infrared agents, chemiluminescent agents, microparticles or nanoparticles (e.g., quantum dots, nanocrystals, semiconductor particles, nanoparticles, nanobubbles, or nanochains and the like), colorimetric labels, magnetic labels, and chelating agents.

In some embodiments, compounds including the detectable moiety described herein may be used in conjunction with non-invasive imaging (e.g., neuroimaging) techniques for in vivo imaging of the compound, such as magnetic resonance spectroscopy (MRS) or imaging (MRI), or gamma imaging, such as positron emission tomography (PET) or single-photon emission computed tomography (SPECT). The term “in vivo imaging” refers to any method, which permits the detection of a labeled compound, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of the compound along with a large excess of unlabeled, but otherwise chemically identical compound.

For purposes of in vivo imaging, the type of detection instrument available is a major factor in selecting a given detectable moiety. For instance, the type of instrument used will guide the selection of the stable isotope. The half-life should be long enough so that it is still detectable at the time of maximum uptake by the target, but short enough so that the host does not sustain deleterious effects.

In one example, the detectable moiety can include a radiolabel, that is directly or indirectly linked (e.g., attached or complexed) with a compound described herein using general organic chemistry techniques. The radiolabel can be, for example, ⁶⁸Ga, ¹²³I, ¹³¹I, ¹²⁵I, ¹⁸F, ¹¹C, ⁷⁵Br, ⁷⁶Br, ¹²⁴I, ¹³N, ⁶⁴Cu, ³²P, ³⁵S. Such radiolabels can be detected by PET techniques, such as described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The detectable moiety can also include ¹²³I for SPECT. The ¹²³I can be coupled to the compound by any of several techniques known to the art. In addition, the detectable moiety can include any radioactive iodine isotope, such as, but not limited to ¹³¹I, ¹²⁵I, or ¹²³I. The radioactive iodine isotopes can be coupled to the compound, for example, by conversion of a non-radioactive halogenated precursor to a stable tri-alkyl tin derivative which then can be converted to the iodo compound by several methods well known to the art.

The detectable moiety can further include known metal radiolabels, such as Technetium-99m (99mTc), ¹⁵³Gd, ¹¹¹In, ⁶⁷Ga, ²⁰¹Tl, ⁸²Rb, ⁶⁴Cu, ⁹⁰Y, ¹⁸⁸Rh, T (tritium), ¹⁵³Sm, ⁸⁹Sr, and ²¹¹At. Modification of the compound to introduce ligands that bind such metal ions can be effected without undue experimentation by one of ordinary skill in the radiolabeling art. The metal radiolabeled compounds can then be used to detect cancers, such as GBM in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2): 135-40, (1998); and Hom et al., “Technetium-99m-labeled receptor-specific small-molecule radiopharmaceuticals: recent developments and encouraging results” Nuclear Medicine & Biology 24(6):485-98, (1997).

In some embodiments, the detectable moiety can include a chelating agent (with or without a chelated radiolabel metal group). Examples chelating agents can include those disclosed in U.S. Pat. No. 7,351,401, which is herein incorporated by reference in its entirety. In some embodiments, the chelating agent is 1,4,7,10-tetraazacyclododecane-1,4,7,10-tetraacetic acid (DOTA).

Fluorescent labeling agents or infrared agents include those known to the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, ALEXA 647, IR700, IR 800, TEXAS RED, and QUASAR 670.

In some embodiments, the detectable moiety includes a fluorescent dye. Examples of fluorescent dyes include fluorescein isothiocyanate, cyanines, such as Cy5, Cy5.5 and analogs thereof (e.g., sulfo-Cyanine 5 NHS ester and Cy5.5 maleimide). See also Handbook of Fluorescent Probes and Research Chemicals, 6th Ed., Molecular Probes, Inc., Eugene Oreg, which is incorporated herein by reference.

The detectable moiety can further include a near infrared imaging group. Near infrared imaging groups are disclosed in, for example, Tetrahedron Letters 49(2008) 3395-3399; Angew. Chem. Int. Ed. 2007, 46, 8998-9001; Anal. Chem. 2000, 72, 5907; Nature Biotechnology vol 23, 577-583; Eur Radiol (2003) 13:195-208; and Cancer 67:1991 2529-2537, which are herein incorporated by reference in their entirety. Applications may include the use of a NIRF (near infra-red) imaging scanner. In one example, the NIRF scanner may be handheld. In another example, the NIRF scanner may be miniaturized and embedded in an apparatus (e.g., micro-machines, scalpel, neurosurgical cell removal device).

Quantum dots, e.g., semiconductor particles, can be employed as detectable moieties as described in Gao, et al “In vivo cancer targeting and imaging with semiconductor quantum dots”, Nature Biotechnology, 22, (8), 2004, 969-976, the entire teachings of which are incorporated herein by reference. The disclosed compounds can be coupled to the quantum dots, administered to a subject or a sample, and the subject/sample examined by fluorescence spectroscopy or imaging to detect the labeled compound.

In certain embodiments, a detectable moiety includes an MRI contrast agent. MRI relies upon changes in magnetic dipoles to perform detailed anatomic imaging and functional studies. MRI can employ dynamic quantitative T1 mapping as an imaging method to measure the longitudinal relaxation time, the T1 relaxation time, of protons in a magnetic field after excitation by a radiofrequency pulse. T1 relaxation times can in turn be used to calculate the concentration of a molecular probe in a region of interest, thereby allowing the retention or clearance of an agent to be quantified. In this context, retention is a measure of molecular contrast agent binding.

Numerous magnetic resonance imaging (MRI) contrast agents are known to the art, for example, positive contrast agents and negative contrast agents. The disclosed compounds can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iron oxide, or the like. Typical contrast agents include macrocycle-structured gadolinium (III) chelates, such as gadoterate meglumine (gadoteric acid), gadopentetate dimeglumine, gadoteridol, mangafodipir trisodium, gadodiamide, and others known to the art. In certain embodiments, the detectable moiety includes gadoterate meglumine. Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

In some embodiments, the compound can be coupled or linked to a chelating agent, such as macrocyclic chelator DOTA, and a single metal radiolabel.

The compounds described herein can be used in a pharmaceutical composition to detect and/or treat a variety of cancers that express RPTP including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoictic tumors of myeloid lineage, including acute and chronic myelogenous leukemias and promyclocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma.

In certain embodiments, cancer cells that express an RPTP can include glioma cells. The term glioma, as used herein, refers to a type of cancer arising from glial cells in the brain or spine. Gliomas can be classified by cell type, by tumor grade, and by location. For example, ependymomas resemble ependymal cells, astrocytmoas (e.g., glioblastoma multiforme) resemble astrocytes, oligodedrogliomas resemble oligodendrocytes. Also mixed gliomas, such as oligoastrocytomas may contain cells from different types of glia. Gliomas can also be classified according to whether they are above or below a membrane in the brain called the tentorium. The tentorium separates the cerebrum, above, from the cerebellum, below. A supratentorial glioma is located above the tentorium, in the cerebrum, and occurs mostly in adults whereas an infratentorial glioma is located below the tentorium, in the cerebellum, and occurs mostly in children.

In still other embodiments, the cancer cells that are detected and/or treated can include invasive, dispersive, motile or metastatic cancer cells, such as invasive, dispersive, motile or metastatic glioma cells, lung cancer cells, breast cancer cells, prostate cancer cells, and melanoma cells. It will be appreciated that other cancer cells and/or endothelial cells, which support cancer cell survival, that express an RPTP cell adhesion molecule and that can be proteolytically cleaved to produce a detectable extracellular fragment can be identified or determined by, for example, using immunoassays that detect the RPTP cell adhesion molecule expressed by the cancer cells or endothelial cells.

A pharmaceutical composition that includes a compound described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the compound probe can be by intravenous injection of the compound in the subject. Single or multiple administrations of the compound can be given. “Administered”, as used herein, means provision or delivery of compound in an amount(s) and for a period of time(s) effective to label or treat cancer cells in the subject.

In some embodiments, the compounds described herein can be administered to a cancer cell, e.g., glioblastoma multiforme cell, prostate cancer, lung cancer, melanoma, or tumor-derived endothelial cell of a subject by contacting the cell of the subject with a pharmaceutical composition described above. In one aspect, a pharmaceutical composition can be administered directly to the cell by direct injection. Alternatively, the pharmaceutical composition can be administered to the subject systematically by parenteral administration, e.g., intravenous administration or oral.

In a further example, the compound can be used in combination and adjunctive therapies for inhibiting cancer cell proliferation, growth, and motility. The phrase “combination therapy” embraces the administration of the compounds described herein and an additional therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). The phrase “adjunctive therapy” encompasses treatment of a subject with agents that reduce or avoid side effects associated with the combination therapy of this application.

A combination therapy is intended to embrace administration of these therapeutic agents in a sequential manner, that is, wherein different therapeutic agents are administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of therapeutic agents can be effected by an appropriate routes including, but not limited to, oral routes, intravenous routes, intramuscular routes, and direct absorption through mucous membrane tissues. The therapeutic agents can be administered by the same route or by different routes. The sequence in which the therapeutic agents are administered is not narrowly critical.

Combination therapy also can embrace the administration of the therapeutic agents as described above in further combination with other biologically active ingredients (such as, but not limited to, a second and different therapeutic agent) and non-drug therapies (such as, but not limited to, surgery or radiation treatment). Where the combination therapy further comprises radiation treatment, the radiation treatment may be conducted at a suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments the compounds described herein can be administered in combination at least one anti-proliferative agent selected from a chemotherapeutic agent, an antimetabolite, an antitumorgenic agent, an antimitotic agent, an antiviral agent, an antineoplastic agent, an immunotherapeutic agent, or a radiotherapeutic agent.

The phrase “anti-proliferative agent” can include agents that exert antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

In some embodiments, a compound including or linked to a detectable can be used in a method to detect and/or determine the presence, location, and/or distribution of cancer cells, i.e., cancer cells associated with RPTP cell adhesion molecules, in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the compound in the animal's tissue, e.g., brain tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the agent may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the composition. In this case, the baseline and post-administration images can be compared to ascertain the presence, absence, and/or extent of a particular disease or condition.

In one aspect, the compound including the detectable moiety may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination. For example, in glioma (brain tumor) surgery, the compound can be given intravenously about 24 hours prior to pre-surgical stereotactic localization MRI. The compounds can be imaged on gradient echo MRI sequences as a contrast agent that localizes with the glioma.

Compounds described herein that include a detectable moiety and specifically bind to and/or complex with RPTP cell adhesion molecules (e.g., PTPμ) expressed by cells or cancer cells can be used in intra-operative imaging (IOI) techniques to guide surgical resection and eliminate the “educated guess” of the location of the tumor margin by the surgeon. Previous studies have determined that more extensive surgical resection improves patient survival Stummer W, Novotny A, Stepp H, Goetz C, Bise K, Reulen HJ (2000) Fluorescence-guided resection of glioblastoma multiforme by using 5-aminolevulinic acid-induced porphyrins: a prospective study in 52 consecutive patients. J Neurosurg 93:1003-1013. Fluorescence-guided resection of glioblastoma multiforme by using 5-aminolevulinic acid-induced porphyrins: a prospective study in 52 consecutive patients. Stummer W, Novotny A, Stepp H, Goetz C, Bise K, Reulen HJ (2000) Fluorescence-guided resection of glioblastoma multiforme by using 5-aminolevulinic acid-induced porphyrins: a prospective study in 52 consecutive patients. J Neurosurg 93:1003-1013. Thus, compounds that function as diagnostic imaging agents have the potential to increase patient survival rates.

In some embodiments, to identify and facilitate removal of cancer cells, microscopic intra-operative imaging (IOI) techniques can be combined with systemically administered or locally administered compounds described herein. The compounds upon administration to the subject can target and detect and/or determine the presence, location, and/or distribution of cancer cells, i.e., cancer cells expressing RPTP cell adhesion molecules, in an organ or body area of a patient. In one example, the compound can be combined with IOI to identify malignant cells that have infiltrated and/or are beginning to infiltrate at a tumor brain margin. The method can be performed in real-time during brain or other surgery. The method can include local or systemic application of the compound described herein that includes a detectable moiety, e.g., a fluorescent or MRI contrast moiety. An imaging modality can then be used to detect and subsequently gather image data. The imaging modality can include one or combination of known imaging techniques capable of visualizing the compound. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

In one example, an agent including a compound linked to a fluorescent detectable moiety can be topically applied as needed during surgery to interactively guide a surgeon and/or surgical instrument to remaining abnormal cells. The compound may be applied locally in low concentration, making it unlikely that pharmacologically relevant concentrations are reached. In one example, excess material may be removed (e.g., washed off) after a period of time (e.g., incubation period).

In certain embodiments, the methods and compounds described herein can be used to measure the efficacy of a therapeutic administered to a subject for treating a metastatic, invasive, or dispersed cancer. In this embodiment, the compound can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the compound can be used to define the distribution of the metastatic cancer pre-operative and post-operative to determine the efficacy of the surgical resection. Optionally, the methods and compounds can be used in an intra-operative surgical procedure as describe above, such as a surgical tumor resection, to more readily define and/or image the cancer cell mass or volume during the surgery.

The compounds described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The compounds described herein can be administered alone as a monotherapy, or in conjunction with or in combination with one or more additional therapeutic agents. For example, the compounds described herein can be administered to the subject prior to, during, or post administration of an additional therapeutic agent and the distribution of metastatic cells can be targeted with the therapeutic agent. The agent can be administered to the animal as part of a pharmaceutical composition comprising the agent and a pharmaceutically acceptable carrier or excipient and, optionally, one or more additional therapeutic agents. The compound described herein and additional therapeutic agent can be components of separate pharmaceutical compositions, which can be mixed together prior to administration or administered separately. The compounds described herein, for example, be administered in a composition containing the additional therapeutic agent, and thereby, administered contemporaneously with the agent. Alternatively, the compounds described herein can be administered contemporaneously, without mixing (e.g., by delivery of the agent on the intravenous line by which the therapeutic agent is also administered, or vice versa). In another embodiment, the compounds described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The compounds described herein (or composition containing the compounds) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the compounds and/or an additional therapeutic agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the compound in the subject, the agent can be administered between, for example, once a day or once a week.

For example, the administration of the compound and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The compounds described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of the compound described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the compound described herein can also depend on the disease state or condition being treated along with the clinical factors and the route of administration of the compound.

The following example is included to demonstrate preferred embodiments.

### Example

We utilized ATOMNET, the first deep learning neural network for structure-based drug design and discovery, developed by Atomwise Inc. to identify small molecules predicted to interact with the extracellular domain of PTPμ and tested them in two cell-based assays: PTPμ-mediated Sf9 aggregation as well as glioma cell sphere formation and three-dimensional (3D) survival and growth. Growth in 3D culture is a characteristic of tumor cells and can be used as a surrogate measure for tumor formation. Our top hit was confirmed to affect PTPμ activity in an in vitro bead-based adhesion assay, indicating the effectiveness of this modeling and screening approach.

### Materials and Methods

### PTPμ-Dependent Sf9 Aggregation Assays

This assay tests cell-cell adhesion or aggregation of cells induced by PTPμ expression. 48-well culture plates (Corning #353078, Corning, NY, USA) were treated with 0.75% (wt/vol) PVA (Sigma-Aldrich, >99+% hydrolyzed, number-average MW ca. 130 kD) to create a unique non-adhesive substrate. Sf9 cells from ATCC cultured in Grace's Complete Medium (Gibco, Grand Island, NY, USA)+10% FBS (HyClone, South Logan, UT, USA) were infected with baculovirus to express human full-length PTPμ. Approximately 40 h after infection, cells were resuspended in the culture media (which includes floating cell clumps detached during infection) and gently triturated to create a single cell suspension. Cells were counted and diluted to a concentration of 12.8×10⁴ cells/mL, and 90 μL (1.15×10⁴) were plated per non-adherent well. To give the indicated final concentrations, 90 μL of compound (prepared in media) was added to the wells (2× replicates per com-pound). Bubbles (which interfere with aggregation) were removed by puffing air across the plate with an empty squeeze bottle. Plates were incubated at room temperature for 20 min, and aggregation was induced by rotation at 120 rpm on an orbital shaker. Plates were manually shaken to distribute aggregates away from the center of the wells; then, each well was imaged in its entirety by capturing a 4×4 grid at 5× magnification on a Leica CTR6500 microscope fitted with an automated stage.

### Glioma Sphere Assays

The same unique PVA substrate described above was used to induce formation of 3D tumor spheroids. As previously described, 96-well u-bottom non-tissue culture-treated plates were coated with 0.75% (wt/vol) PVA and allowed to dry upside down. LN229 cells, obtained from ATCC, were cultured to confluence in DMEM (High Glu-cose DMEM (Gibco, Grand Island, NY, USA)+5% FBS (HyClone, South Logan, UT, USA), trypsinized, and resuspended at a plating concentration of 15×10⁴ cells/mL, and 50 μL (7500 cells) plated per well into the internal wells of the prepared plate. To give the indicated final concentrations, 50 μL of compound (prepared in media) was added to the wells (2× replicates per compound) and 100 μL of PBS was plated per well into the external wells of the plate (to buffer edge effects), and the plate was cultured for 7 days at 37° C. and 5% CO₂. Spheres were imaged at 5× magnification using a Leica CTR6500 microscope fitted with an automated stage.

### Bead Aggregation Assay

Bead aggregation assays were performed in 48-well plates pretreated with anti-adherence rinse (Stemcell Technologies, Vancouver, CA, USA) per the manufacturer's instructions. A bacterially expressed biotin-tagged fragment of PTPμ corresponding to aa 21-365 (PTPμ21_365avi—containing the MAM, Ig, and first FNIII repeat) was diluted into PBS+0.01% Tween 20 to 30 ng/μL. Streptavidin MonoMag beads (1 micron diameter, Ocean NanoTech, San Diego, CA, USA) were added at a ratio of 53 ng protein/μg of beads and incubated at room temperature for 15 min. Coated beads were diluted to 6 μg beads/mL (~1.6×10⁷ particles/mL) in PBS+0.01% Tween 20, and 90 μL used per well. Compounds were diluted into PBS+0.01% Tween 20 and 90 μL added per well (2× replicates) to give a final concentration of 100 μM. The plate was incubated at room temperature for 20 min; then, aggregation was induced by rotation at 150 rpm for 30 min. The plate was manually shaken to randomly distribute aggregates, and sixteen 20× images (a 4×4 grid with 1000 micron spacing in both x- and y-axes) captured per well on a Leica CTR6500 microscope fitted with an automated stage. This does not tile the entire well, but captures a sampling to account for the random distribution of particles.

### Image Analysis

The number of Sf9 aggregates over an arbitrary footprint-size threshold of 4000 μm2 were quantified using ImageJ.

The number of smoothing steps can be adjusted as necessary to yield uniform aggregate outlines. The upper limit of size was applied to eliminate flagging large clusters of loose cells (which often swirl to the center) as an aggregate. This macro was created to accommodate illumination differences between the edge and center of the plate; however manual correction was necessary to detect shadowed aggregates, adjust for touching aggregates or pooling of loose cells, eliminate debris etc.

The footprint size of spheres was measured with ImageJ:

Data for each replicate were normalized to the average value of the DMSO controls and then averaged and presented as a percentage±standard error of the mean. The majority of compounds were only assessed with an n of two, but for compounds flagged as active in the initial screen, the n varies from 4-8 replicates at 100 μM and 2 replicates at 50 μM and 25 μM.

### Results

### Atomwise Virtual Screen

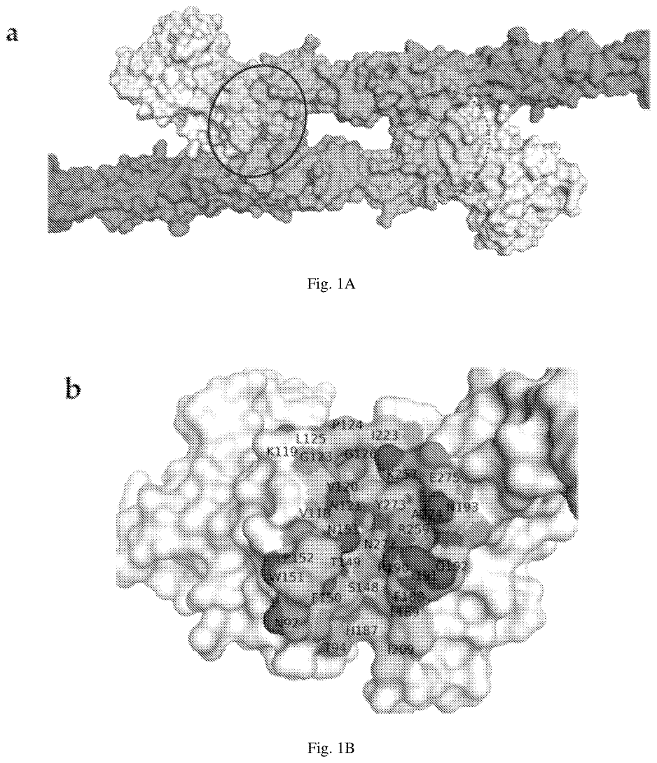

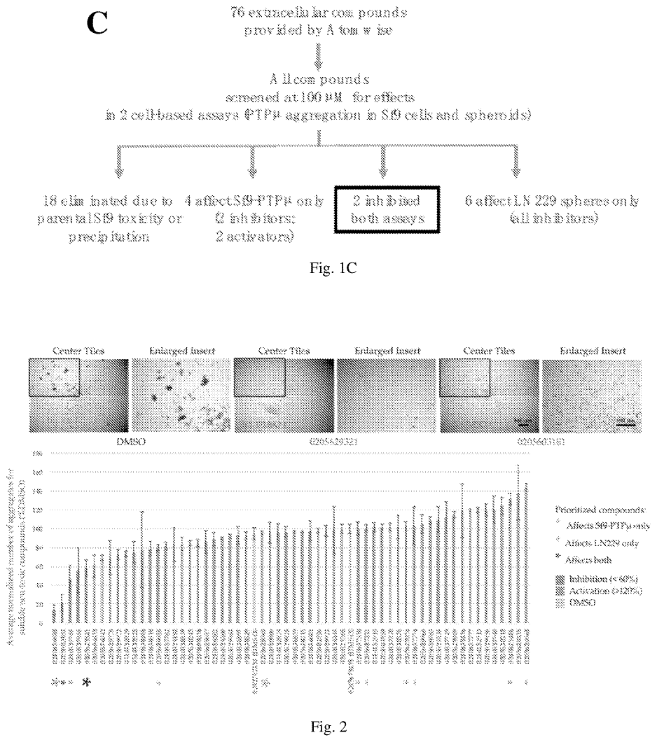

We investigated the available crystal structures for PTPμ and related proteins to identify suitable binding sites for small molecules. The groove involved in the trans-dimer interaction seen in the crystal structure [Protein Data Bank IDs 2V5Y] is predicted to be the most druggable site on the extracellular domain (ECD) (FIGS. 1A, B) based on the ICM pocket finder module (v3.8-7, Molsoft L.L.C. (San Diego, CA, USA)), which assigns a druggability score based on the volume of the putative binding site and its hydrophobicity and the degree to which the site is buried. This pocket sits between the MAM and Ig domains and partially overlaps with the homodimer contact site. Therefore, targeting it could restrict flexibility between those two critical domains and prevent certain types of homophilic interactions. Thus, Atomwise selected this region as the target site for virtual high throughput screening.

A single global ATOMNET model, which is a tool for structure-based drug design and predicting protein-ligand interactions, was used to predict the binding affinity of small molecules to the putative PTPμ drug-binding pocket. The binding constants (i.e., Kᵢ, K_{d}, and IC₅₀ values), based on experimental data, the structures of thousands of proteins from various families, and millions of small molecules all derived from curated public databases and proprietary sources, were used to train the model that was then used to screen for novel binding sites and ligands. Because ATOMNET is a single global model, the likelihood of overfitting is reduced. The model training used a three-step procedure: (1) a flooding algorithm based on an initial seed was used to define the binding site, with the seed derived from either bound ligands annotated in the PDB database, previously reported catalytic motifs, or critical residues revealed through mutagenesis; (2) the coordinates of the protein-ligand complex were translated into a 3D Cartesian space with the origin defined as the center-of-mass of the binding site. The protein structure was randomly rotated and translated around the center-of-mass of the binding site to prevent the neural network from memorizing a preferred structural orientation; (3) for each ligand, multiple poses, each representing a putative protein-ligand complex, within the binding site were sampled, which means that our method does not require experimental co-complexes for either training or prediction. Uniformly-sized regular 3D grids were generated by rasterizing each co-complex, with grid point values representing the presence of different atom types at that point, similar to how photo imaging assigns pixels into red, green, and blue channels. The receptive field of our convolutional neural network is defined by these grids. The network architecture was as described in Hsieh et al. Final scores for each binding pose were calculated through weighted Boltzmann averaging and compared to the experimentally measured pKa, pKi, or pIC50 of the protein and ligand pair. A root-mean-square-error loss function was used to adjust the weights of the neural network, thus, reducing the error between predicted and experimentally measured affinities. Training was done with the ADAM adaptive learning method using the backpropagation algorithm and mini-batches with 64 examples per gradient step.

The trained ATOMNET model was used to score and rank compounds from the 20180722 version of the Mcule library of commercially available small drug-like molecules (6.8 M compounds after pre-processing, https://mcule.com/, accessed on 1 Oct. 2018) using an ensemble of ligand-protein conformations. The 30,000 top-scoring compounds were filtered to remove compounds that were predicted to be insoluble by Atomwise's solubility model or contained undesired (potentially reactive, unstable, or promiscuous) chemical moieties. The Butina clustering algorithm, which selects compounds by going down a ranked list and keeping only those entries that meet a certain diversity cutoff (here: ECFP4 fingerprint similarity with a Tanimoto coefficient≥0.4) was employed to arrive at a final subset of 77 deliverable compounds. These compounds were provided together with two blinded DMSO controls.

### Functional Screening Strategy

The 76 compounds predicted by computational modeling to interact with the extracellular domain of PTPμ were screened in two cell-based assays: an Sf9 aggregation assay and a glioma cell 3D sphere formation and growth assay (FIG. 1C). Two DMSO samples were provided by Atomwise as blinded controls, while a third non-blinded DMSO sample was used as a control for normalization purposes. The Sf9 assay was chosen because it directly tests the adhesive action of PTPμ since Sf9 cells lack endogenous PTPμ (and other RPTPIIb family members) and do not normally self-aggregate. However, baculoviral-mediated overexpression of PTPμ drives homophilic adhesion of Sf9 cells. Historically, aggregation assays were performed in glass scintillation vials, which have ideal surface characteristics but are unwieldy for screening purposes. This assay was scaled to a low volume multi-well format to accommodate moderate throughput. We achieved this by adjusting rotation speed and utilizing an affordable and adaptable non-adhesive coating (highly hydrolyzed polyvinyl alcohol) that we developed. Without this coating, cells stick to the plastic (whether tissue-culture treated or not) and fail to aggregate.

We used the same versatile non-adhesive coating to perform a glioma sphere-based assay. This assay was selected to run in parallel because the ultimate goal is to identify compounds that have therapeutic potential against cancers, such as glioblastoma. Glioma cells (LN229s) cultured on our non-adhesive coating cluster together and grow as 3D structures that can model some of the complexity of the tumor microenvironment. In particular, a peptide imaging agent that recognizes the extracellular domain of PTPμ labels the central core of these spheres, indicating the possible accumulation of cleaved fragments, similar to what is observed in actual tumors.

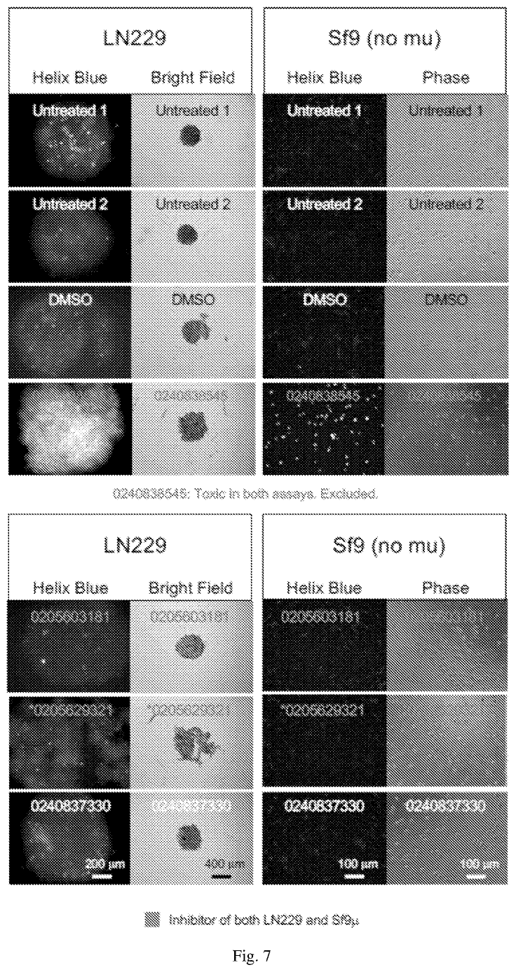

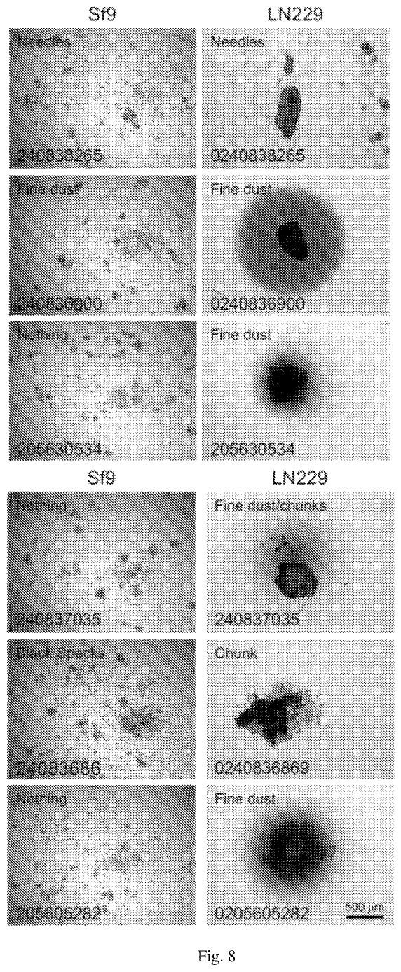

Eighteen compounds were eliminated due to either general toxicity or poor solubility. One compound was found to be toxic to parental Sf9 cells, indicating an off-target effect as these cells lack expression of PTPμ (FIG. 7), and 17 compounds were eliminated due to insolubility under the conditions used for the Sf9 aggregation assay and/or the 3D sphere assay (FIG. 8). The precipitates appeared as fine dust accumulating around the spheres (due to the u-bottom shape of the plate), or as specks, needles, scattered debris, etc.

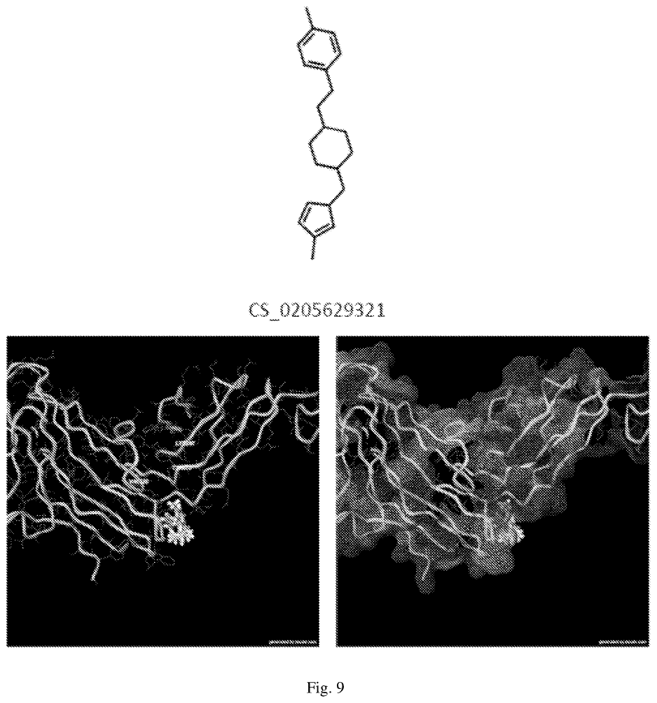

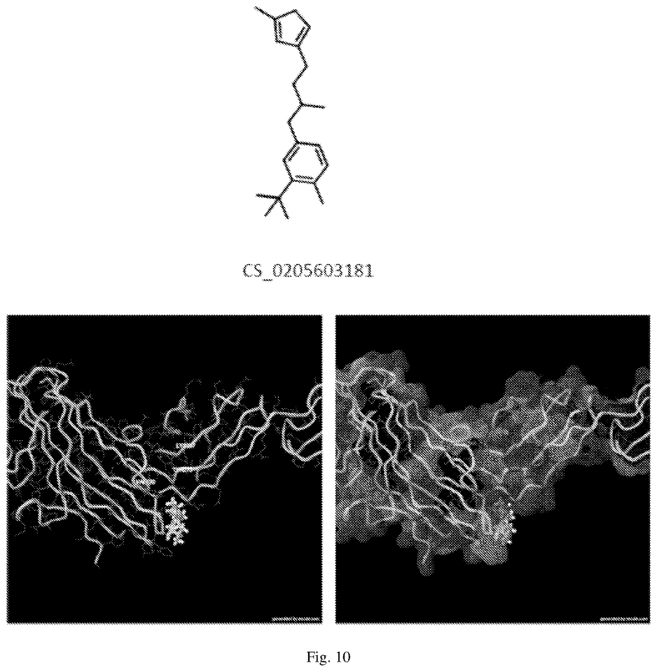

Of the remaining 58 compounds, four affected PTPμ aggregation only, six affected glioma 3D spheres (aggregation and/or growth) only, and two, our priority compounds, were inhibitors in both assays. The predicted Gibb's free energy for association of 0205629321 was −6.0. The predicted Gibb's free energy for 0205603181 was −6.3. Carbon-substituted base structures and binding poses of the two priority compounds are shown in FIGS. 9 and 10.

### PTPμ-Dependent Sf9 Aggregation

FIG. 2 shows examples of the effects of our priority compounds (bar codes 0205629321, 0205603181) on PTPμ-mediated aggregation of Sf9 cells. Aggregates are readily apparent in control samples, but wells treated with putative PTPμ inhibitors contain mostly single cells or small clusters.

The effects of the compounds on PTPμ-dependent aggregation were quantified by counting the total number of aggregates above 4000 μm2 in each well and normalizing that to the average number present in the un-blinded vehicle control wells. FIG. 2 shows the results for all the soluble non-toxic compounds. In addition to our two priority compounds, we identified compounds that affected PTPμ aggregation only (positions indicated by color-coded asterisks). The strongest two inhibitors (0240836885 and 0240837785) and one activator (0240821486) in this category were selected for further analysis. Compounds exhibiting poor reproducibility between replicates were rejected. As indicated in FIG. 1, we also identified compounds that affected tumor spheres without affecting PTPμ aggregation, and the positions of these compounds (color-coded asterisks) are indicated for reference.

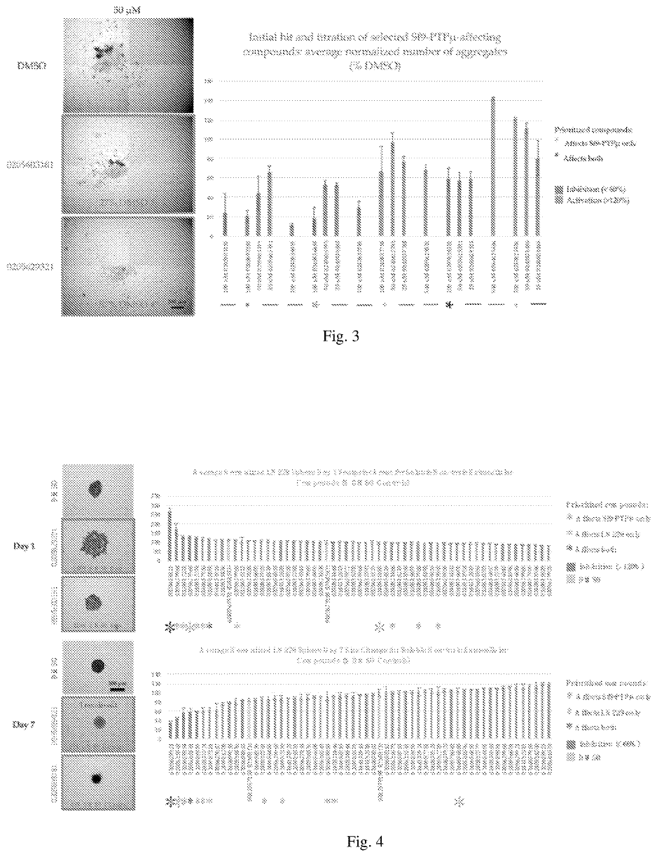

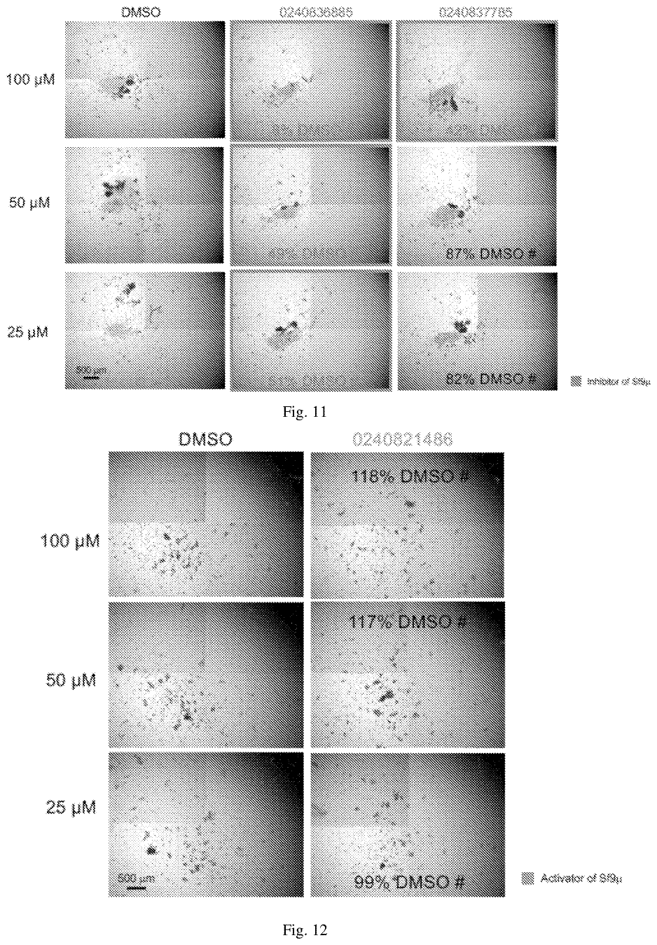

Our initial screen was performed at 100 μM, but to identify the most potent agents we titrated our two priority compounds and other selected hits (FIGS. 3, 11 and 12). Of the two priority inhibitors, only one (0205629321) was active down to 25 μM and in fact, exhibited the same degree of inhibition (~60%) at all tested doses. This could indicate that we have saturated binding with this compound at all tested concentrations, and that even with 100% occupancy the compound cannot fully abolish the adhesive activity of PTPμ. It is likely that its activity will taper at lower doses, but these have not yet been tested. Of the Sf9-PTPμ-only inhibitors, one compound (0240836885, the strongest identified in FIG. 2) was also active down to 25 μM (FIG. 3). The one tested activator was weaker at 100 μM on follow-up and was not effective at lower doses (FIGS. 3 and 12).

### Glioma Sphere Formation and Growth

When glioma cells are cultured on non-adherent surfaces, they compact into an aggregate and grow as a 3D structure. We tested all soluble non-toxic compounds in this assay, and FIG. 4 shows representative effects of our two priority compounds on glioma cell aggregation and consolidation (day 1) and sphere growth (day 7). The two compounds slowed both of these processes, resulting in a broader spread of cells on day 1 and smaller spheres by day 7. One of the priority compounds (0205629321) also produced an interesting qualitative change in the appearance of the day 7 spheres. These spheres were translucent instead of optically dense.

To quantify sphere aggregation, we measured the day 1 footprint areas of the spheres and normalized that to the area of the vehicle-treated controls (FIG. 4). To quantify sphere growth, we determined the change in footprint size between day 1 and day 7 and normalized that to the size change of the controls (FIG. 4). Of our two priority compounds (color-coded asterisks), the one with the most potent effect on PTPμ aggregation (0205629321) based on it retaining activity down to 25 μM (FIG. 3) was also the strongest in these assays. The other priority inhibitor (0205603181) affected sphere growth but not aggregation. As indicated in FIG. 1, there were also six compounds that were ineffective in the PTPμ aggregation assay but behaved as inhibitors of glioma sphere condensation and/or growth (color-coded asterisks in FIG. 4). The positions of the four compounds that affected PTPμ aggregation but not glioma spheres are also shown for reference.

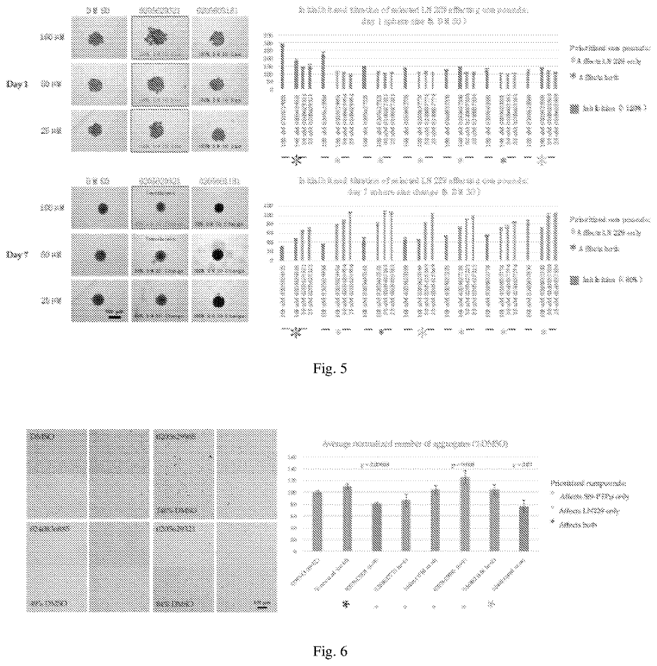

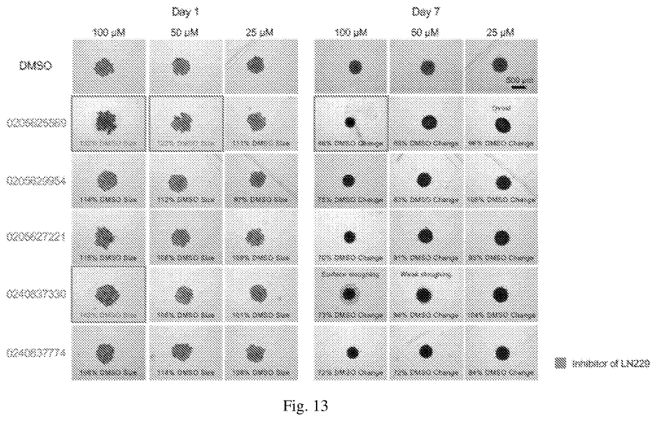

We titrated the priority compounds (FIG. 5) and five select glioma sphere-only inhibitors (FIGS. 5 and 13) to identify the compounds with the most potent effects on glioma cells. Our best priority candidate (0205629321) affected sphere aggregation down to 25 μM but was only effective on sphere growth at 100 μM. A change in sphere opacity was, however, observed down to 25 μM. The other priority compound did not affect sphere condensation in follow-up and had only very modest effects on sphere growth. The compounds identified as affecting glioma spheres only were either weak or lacked activity in follow-up. Two of these compounds did, however, cause interesting qualitative changes in the day 7 appearance of spheres (FIG. 13). Spheres exposed to 25 μM of one of these compounds (0205625569) were ovoid instead of round. In addition, one compound (0240837330) appeared to cause sloughing of cells from the surface of spheres when used at both 50 UM and 100 μM. Based on Helix Blue staining, this compound was not acutely toxic (at 24 h of treatment) (FIG. 7) but the sloughing of cells on day 7 could indicate long-term toxicity.

### Bead Assay to Assess Direct Interactions Between Selected Compounds and PTPμ

Because off-target effects are possible in cell-based assays, we tested our priority compounds (that affected both PTPμ aggregation in Sf9 cells and inhibited glioma spheroids) and select compounds that just affected PTPμ aggregation in Sf9 cells in a PTPμ-mediated bead aggregation assay (FIG. 6). One weak compound from the glioma-only category was tested for comparison. Streptavidin beads were coated with a bacterially-expressed avi-tagged fragment of PTPμ containing the MAM, Ig, and first FNIII repeat (PTPμ21-365avi). Coated beads were treated with compounds (100 μM) for 20 min and then induced to aggregate by rotation. One of our priority inhibitors (0205629321) had a modest, but statistically significant, effect on bead aggregation. The other priority inhibitor was not soluble in the buffer used for this assay (PBS+0.01% Tween) despite being soluble in media. Two compounds from the Sf9-only category also had statistically significant effects: 0240836885 inhibited and 0205629965 activated bead aggregation. Notably, 0240836885 was the strongest inhibitor and 0205629965 the strongest activator of Sf9-PTPμ-aggregation identified in the screen (FIG. 2).

Using AI-targeted drug design and cell-based functional screens, we identified compounds able to perturb PTPμ-dependent adhesion and/or affect glioma cell growth in 3D culture. The PTPμ aggregation assay is a direct test of PTPμ-mediated aggregation, as parental Sf9 cells lack PTPμ, so compounds affecting this assay are more likely to be specific targeting agents; however, compounds affecting spheres are more likely to have therapeutic potential. The fact that we obtained inhibitory compounds in the sphere assay that produced qualitatively different effects (i.e., changes in optical appearance and shape or sloughing of cells) indicates that different processes are being perturbed. The “clearing” effect of the priority compound that acted as an inhibitor of both PTPμ-dependent Sf9 aggregation and bead aggregation may serve as a useful benchmark for what to expect when PTPμ-dependent adhesion is perturbed in 3D cancer spheroids.

Identifying agents able to affect the complicated interactions required for efficient adhesion (which in the case of PTPμ involves multiple domains and both trans and cis interactions) is not straightforward. Biochemical assays using fragments cannot model the kinds of interactions seen with the full-length protein in the context of a cell membrane. In this sense, it is surprising, and impressive, that we were able to identify an agent effective in both cell and bead-based binding assays, considering we were targeting a single binding pocket in what is likely a large adhesive interface. However, the agents that affected PTPμ aggregation in Sf9 cells, but not bead aggregation, are still worth pursuing as their inhibition of PTPμ may be more conformationally sensitive. Their structures can inform how to improve the interaction between a compound and PTPμ, and structure-activity relationship studies might yield compounds with higher affinity or better activity in cancer-cell assays. Additionally, small molecules able to interact with PTPμ could be further derivatized to serve as both imaging and therapeutic agents (i.e., theranostics). These could be useful agents for treating glioblastoma and other cancers (breast, prostate, lung, ovarian, endometrial, and melanoma) where PTPμ is proteolytically dysregulated.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of inhibiting receptor protein tyrosine phosphatase (RPTP) mediated adhesion of RPTP expressing cells and/or RPTP expressing cancer cell growth and/or cancer cell sphere formation, the method comprising:
administering to the RPTP expressing cells and/or RPTP expressing cancer cells a pharmaceutical composition comprising a compound of formula (I):
U-V-W-X-Y (I) or a pharmaceutically acceptable salt, tautomer, or solvate thereof and a pharmaceutically acceptable excipient or a carrier, wherein
U is cycloalkyl, aryl, heteroaryl, or heterocyclyl, each of which is optionally substituted with one or more R¹⁴;
V is absent, alkylene, —C(O)—, —C(O)—N(R¹⁵)₂—, or —N(R¹⁵)₂—C(O)—, -alkylene-C(O)—N(R¹⁵)₂—, or —N(R¹⁵)₂—C(O)-alkylene-;
W is alkylene, heterocyclene, heterocyclene-alkylene-N(R¹⁵)₂—, or arylene, each of which is optionally substituted with one or more R¹⁶;
X is absent or S(O)ₙ;
Y is alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O— alkyl, or -alkylene-COOH;
each R¹⁶ is independently halogen, hydroxyl, alkyl, haloalkyl, or alkoxy;
each R¹⁷ is independently halogen, hydroxyl, alkyl, alkoxy, haloalkyl, cycloalkyl, aryl, heteroaryl, or heterocyclyl;
n is 0, 1, or 2; and
t is 0, 1, or 2.

2. The method of claim 1, wherein U is 5- to 10-membered heterocyclyl, 5- to 10-membered heteroaryl, or 6- to 10 membered aryl, each of which is optionally substituted with one or more R¹⁴.

3. The composition method of claim 1, wherein U is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

4. The method of claim 1, wherein W is C₁-C₆ alkylene, 3- to 6-membered heterocyclene, -(3- to 6-membered heterocyclene)-(C₁-C₆ alkylene)-N(R¹⁵)₂—, or 6 to 10 membered arylene, each of which is optionally substituted with one or more R¹⁶.

5. The method of claim 1, wherein X is S(O)₂.

6. The method of claim 1, wherein X is absent.

7. The method of claim 1, wherein Y is C₁-C₆ alkyl, 5- to 10-membered heterocyclyl, 5- to 10-membered heteroaryl, or 6- to 10 membered aryl, each of which is optionally substituted with one or more R¹⁷.

8-14. (canceled)

15. The method of claim 1, wherein the compound is selected from: 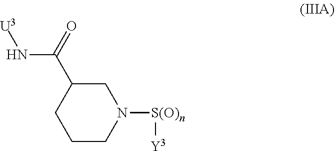
or a pharmaceutically acceptable salt, tautomer, or solvate thereof and a pharmaceutically acceptable excipient or a carrier, wherein
U³ is cycloalkyl, aryl, heteroaryl, or heterocyclyl, each of which is optionally substituted with one or more R¹⁴;
Y³ is alkyl, cycloalkyl, aryl, heteroaryl, heterocyclyl, each of which is optionally substituted with one or more R¹⁷;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁵ is independently, H, alkyl, -alkylene-OH, —C(O)-alkyl, —C(O)O— alkyl, or -alkylene-COOH;
each R¹⁷ is independently halogen, hydroxyl, alkyl, alkoxy, haloalkyl, cycloalkyl, aryl, heteroaryl, or heterocyclyl; and
n is 0, 1, or 2.

16. The method of claim 15, wherein U³ is 5- to 10-membered heterocyclyl, 5- to 10-membered heteroaryl, or 6- to 10 membered aryl, each of which is optionally substituted with one or more R¹⁴.

17. The method of claim 15, wherein U³ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

18. The method of claim 15, wherein Y³ is C₁-C₆ alkyl, 5- to 10-membered heterocyclyl, 5- to 10-membered heteroaryl, or 6- to 10 membered aryl, each of which is optionally substituted with one or more R¹⁷.

19-27. (canceled)

28. The method of claim 1, wherein the compound is selected from: 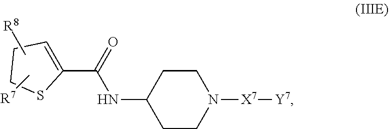
or a pharmaceutically acceptable salt, tautomer, or solvate thereof and a pharmaceutically acceptable excipient or a carrier, wherein
X⁷ is absent or S(O)ₙ;
Y⁷ is alkyl, cycloalkyl, aryl, heteroaryl, heterocyclyl, each of which is optionally substituted with one or more R¹⁷;
each of R⁷ or R⁸ is independently hydrogen, halogen, alkyl, or haloalkyl, or alternatively R⁷ and R⁸ together with the atom(s) to which they are attached can form a 4- to 7-membered cycloalkyl, aryl, heteroaryl, or heterocycle, optionally containing an additional heteroatom selected from O, S(O)ₜ, or N;
each R¹⁷ is halogen, hydroxyl, alkyl, alkoxy, haloalkyl, cycloalkyl, aryl, heteroaryl, or heterocyclyl; and
n is 0, 1, or 2.

29. The method of claim 28, wherein Y⁷ is C₁-C₆ alkyl, 5- to 10-membered heterocyclyl, 5- to 10-membered heteroaryl, or 6- to 10 membered aryl, each of which is optionally substituted with one or more R¹⁷.

30-32. (canceled)

33. The method of claim 1 wherein the compound is selected from: 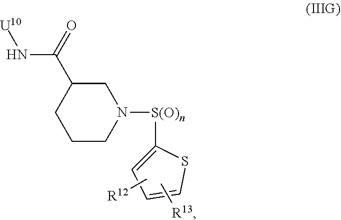
or a pharmaceutically acceptable salt, tautomer, or solvate thereof and a pharmaceutically acceptable excipient or a carrier, wherein
U¹⁰ is independently cycloalkyl, aryl, heteroaryl, or heterocyclyl, each of which is optionally substituted with one or more R¹⁴;
each of R¹² or R¹³ is independently hydrogen, halogen, alkyl, or haloalkyl, or alternatively R¹² and R¹³ together with the atom(s) to which they are attached can form a 4- to 7-membered cycloalkyl, aryl, or heterocycle, optionally containing an additional heteroatom selected from O, S(O)ₜ, or N;
each R¹⁴ is independently —CN, halogen, hydroxyl, alkyl, haloalkyl, alkoxy, aryl, heteroaryl, cycloalkyl, or heterocyclyl, or alternatively two R¹⁴ together with the atom(s) to which they are attached can form a 4- to 7-membered cycloalkyl, aryl, heteroaryl, or heterocycle, optionally containing an additional heteroatom selected from O, S(O)ₜ, or N; and
n is 0, 1, or 2.

34. The method of claim 33, wherein U¹⁰ is 5- to 10-membered heterocyclyl, 5- to 10-membered heteroaryl, or 6- to 10 membered aryl, each of which is optionally substituted with one or more R¹⁴.

35. The method of claim 33, wherein U¹⁰ is thiophenyl, oxazolyl, isoxazolyl, isothiazolyl, thiazolyl, benzothiophenyl, isoxazolyl, diazaindanyl, or phenyl, each of which is optionally substituted with one or more R¹⁴.

36. The method of claim 1, wherein the compound has a structure selected from 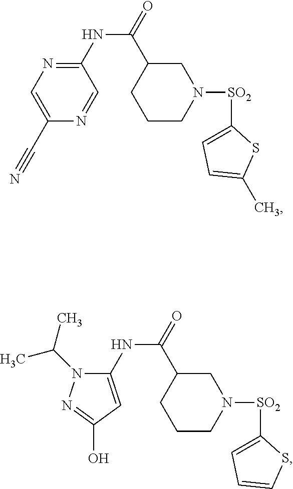 [SMILES: Cc1ccc(S(=O)(=O)N2CCCC(C(=O)Nc3cnc(C#N)cn3)C2)s1 ; CC(C)n1nc(O)cc1NC(=O)C1CCCN(S(=O)(=O)c2cccs2)C1] 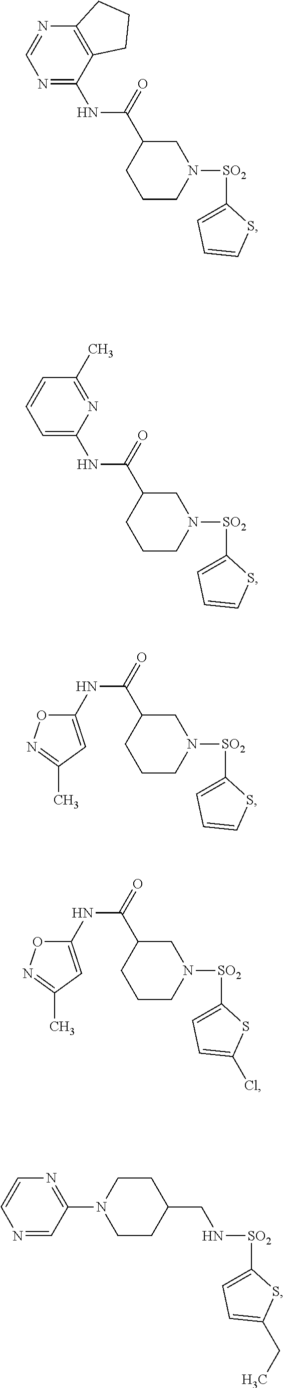 [SMILES: O=C(Nc1ncnc2c1CCC2)C1CCCN(S(=O)(=O)c2cccs2)C1 ; Cc1cccc(NC(=O)C2CCCN(S(=O)(=O)c3cccs3)C2)n1 ; Cc1cc(NC(=O)C2CCCN(S(=O)(=O)c3cccs3)C2)on1 ; Cc1cc(NC(=O)C2CCCN(S(=O)(=O)c3ccc(Cl)s3)C2)on1 ; CCc1ccc(S(=O)(=O)NCC2CCN(c3cnccn3)CC2)s1] 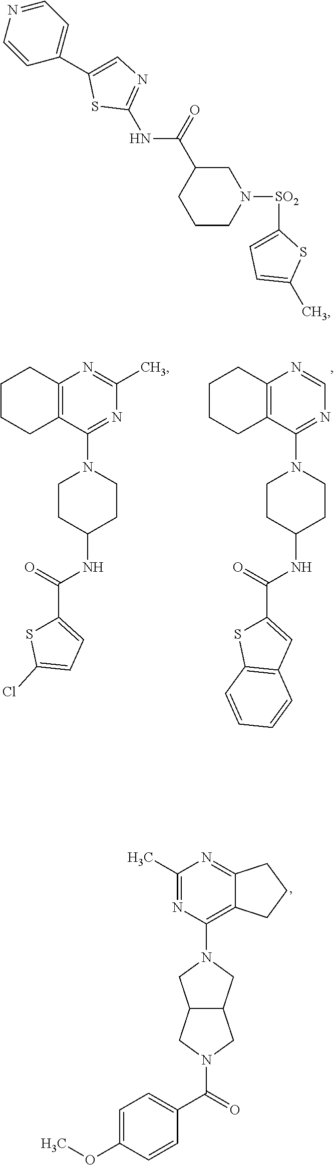 [SMILES: Cc1ccc(S(=O)(=O)N2CCCC(C(=O)Nc3ncc(-c4ccncc4)s3)C2)s1 ; Cc1nc2c(c(N3CCC(NC(=O)c4ccc(Cl)s4)CC3)n1)CCCC2 ; O=C(NC1CCN(c2ncnc3c2CCCC3)CC1)c1cc2ccccc2s1 ; COc1ccc(C(=O)N2CC3CN(c4nc(C)nc5c4CCC5)CC3C2)cc1] 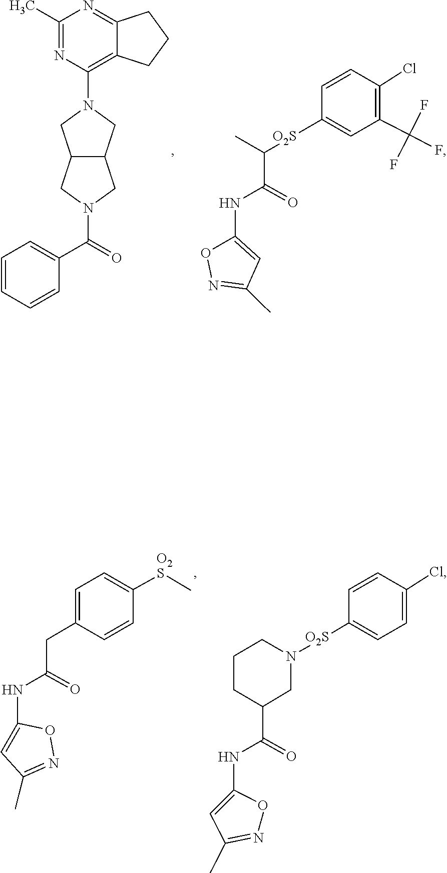 [SMILES: Cc1nc2c(c(N3CC4CN(C(=O)c5ccccc5)CC4C3)n1)CCC2 ; Cc1cc(NC(=O)C(C)S(=O)(=O)c2ccc(Cl)c(C(F)(F)F)c2)on1 ; Cc1cc(NC(=O)Cc2ccc(S(C)(=O)=O)cc2)on1 ; Cc1cc(NC(=O)C2CCCN(S(=O)(=O)c3ccc(Cl)cc3)C2)on1] 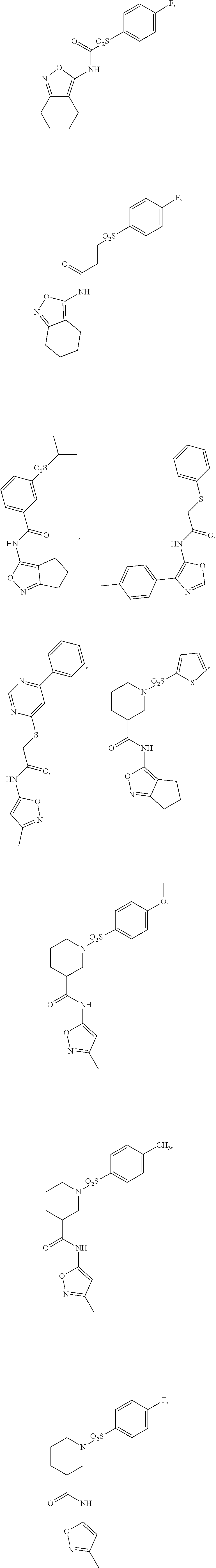 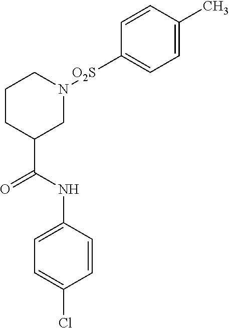 [SMILES: Cc1ccc(S(=O)(=O)N2CCCC(C(=O)Nc3ccc(Cl)cc3)C2)cc1]
or a pharmaceutically acceptable salt, tautomer, or solvate thereof and a pharmaceutically acceptable excipient or a carrier.

37. The method of claim 1, wherein the compound specifically binds to and/or complexes with a RPTP cell adhesion molecule that is expressed by a cancer cell or another cell in the cancer cell microenvironment.

38. The method of claim 1, wherein the pharmaceutical composition is used for treating cancer in a subject in need thereof.

39. (canceled)

40. The method of claim 1, wherein the compound further includes a detectable moiety linked to and/or complexed with compound, the detectable moiety including at least one of a contrast agent, imaging agent, radiolabel, semiconductor particle, or nanoparticle.

41-43. (canceled)
